(19) United States
(12) Patent Application Publication
(10) Pub. No.: US 2021/0255199 A1
(43) Pub. Date: Aug. 19, 2021

(54) PREDICTION OF DRUG-FREE REMISSION IN RHEUMATOID ARTHRITIS

(71) Applicant: University of Newcastle Upon Tyne, Newcastle Upon Tyne (GB)

(72) Inventors: Kenneth Baker, Newcastle Upon Tyne (GB); John Isaacs, Newcastle Upon Tyne (GB); Arthur Pratt, Newcastle Upon Tyne (GB)

(21) Appl. No.: 17/045,428

(22) PCT Filed: Mar. 28, 2019
(86) PCT No.: PCT/GB2019/050902

§ 371 Date: Oct. 5, 2020

(30) Foreign Application Priority Data
Apr. 5, 2018 (GB) 1805685.3

(51) Int. Cl. G01N 33/68; G01N 33/50

(52) U.S. Cl. CPC G01N 33/6893; G01N 33/6863; G01N 2800/54; G01N 2800/102; G01N 33/5005

(57) ABSTRACT

Certain embodiments of the present invention relate to methods and products for use in the determination of treatment of rheumatoid arthritis (RA). Particularly, although not exclusively, embodiments of the present invention relate to predicting the likelihood of a patient suffering from rheumatoid arthritis maintaining remission following cessation of disease-modifying anti-rheumatic drugs (DMARDs). Certain embodiments of the present invention are based on the determination of certain biomarkers of drug-free remission in RA following cessation of DMARD therapy.

### FIELD OF THE INVENTION

Certain embodiments of the present invention relate to methods and products for use in the determination of treatment of rheumatoid arthritis (RA). Particularly, although not exclusively, embodiments of the present invention relate to predicting the likelihood of a patient suffering from rheumatoid arthritis maintaining remission following cessation of disease-modifying anti-rheumatic drugs (DMARDs). Certain embodiments of the present invention are based on the determination of certain biomarkers of drug-free remission in RA following cessation of DMARD therapy.

### BACKGROUND TO THE INVENTION

Rheumatoid arthritis (RA) is a common autoimmune disease with an estimated worldwide prevalence of 0.24% that is characterised by joint inflammation and systemic manifestations. Chronic synovitis causes joint pain, stiffness, swelling and ultimately erosions leading to irreversible joint deformity. In addition to physical disability, additional extra-articular features such as accelerated atherosclerosis add to the excess morbidity and mortality of the disease.

With an increasingly ageing population, the cumulative lifetime risk of RA can be expected to increase over the next few decades. RA inflicts a substantial burden of illness—in 2010 alone, it was the cause of an estimated 3.7 million years lived with disability worldwide. The best-recognised morbidities associated with RA are physical disability and pain resulting from the progressive joint destruction associated with uncontrolled synovitis.

There is no single investigation that is diagnostic of RA, which remains a clinical diagnosis based largely upon medical history and examination findings. Nevertheless, there are several biochemical, serological and radiological investigations that can provide evidence to support the diagnostic process.

The diagnosis of RA has been formalised for research purposes by the creation of classification criteria endorsed by leading rheumatology societies. Until recently, the most widely used of these was the 1987 American College of Rheumatology (ACR) RA classification criteria. Whilst providing a sensitivity and specificity of around 90% for the diagnosis of established RA versus non-RA rheumatic disease controls, the 1987 criteria relied heavily upon features of advanced disease such as radiographic joint damage. Consequently, the 1987 criteria were criticised for their insensitivity to early disease, which made them out-dated with a later evolution towards intervention at earlier stages of clinical presentation. To address this shortfall, the ACR in collaboration with the European League Against Rheumatism (EULAR) released updated RA classification criteria in 2010. The 2010 criteria use a point-based system to define RA across four domains: joint involvement, serological status, biochemical inflammatory markers and symptom duration.

There are two principal aims in the management of RA, namely the alleviation of joint symptoms, and a reduction (ideally prevention) of joint damage and systemic disease manifestations. Long-term outcomes are more favourable when treatment is started early, and when there is comprehensive suppression of inflammation. Indeed, disease remission is now a realistic target of treatment for RA in the modern era.

One class of compounds used for the treatment of RA are so-called disease modifying anti-rheumatic drugs (DMARDs). Many of the early DMARDs including methotrexate, sulphasalazine and hydroxychloroquine are still in widespread use today.

Over the past two decades, increasing knowledge of the molecular basis of inflammation in RA, coupled with technological advances in therapeutic antibody production, have led to the development of novel biopharmaceutical agents. The mechanisms of action of these so-called “biologic” disease modifying anti-rheumatic drugs (bDMARDs) are typified by the potent but selective blockade of an inflammatory mediator, usually by means of a specific antibody or receptor fusion protein. The first of these agents to be licensed for use in RA was infliximab, a chimeric monoclonal antibody directed against tumour necrosis factor α (TNF-α). The following years have seen an explosion in the number of biologic agents, and their potent action has revolutionised the treatment of RA resistant to conventional synthetic DMARD therapy.

Remission is achievable with DMARDs prescribed in modern treat-to-target strategies, albeit with potential side effects and the requirement for safety monitoring. Approximately half of patients with RA who achieve clinical remission with DMARD therapy can expect to achieve sustained drug-free remission (DFR) following DMARD withdrawal. However, there are a lack of biomarkers that, when measured prior to DMARD withdrawal, can predict which patients can achieve DFR.

It is an aim of the present invention to provide biomarkers of drug-free remission in RA to determine when it is appropriate to stop DMARD therapy once remission has been achieved.

### SUMMARY OF CERTAIN EMBODIMENTS OF THE INVENTION

The inventors have identified novel biomarkers of drug-free remission in RA, offering insights to the pathophysiology of arthritis flare. The identification of these biomarkers allows methods to guide DMARD withdrawal, with consequent minimisation of medication side effects and healthcare costs. The identification of these biomarkers allows the development of personalised treatment strategies for RA, and related assays and kits. Accordingly, the invention provides:
A method of determining the likelihood of a patient maintaining remission of rheumatoid arthritis (RA) following cessation of treatment with one or more disease-modifying anti-rheumatic drugs (DMARDs), the method comprising:
   (i) detecting levels of one or more biomarkers in a sample of CD4⁺ T cells obtained from the patient; and
   (ii) comparing the levels obtained in (i) with one or more reference levels;

wherein a difference in levels of the one or more biomarkers compared to the one or more reference levels is indicative of an increased likelihood of maintaining remission following the cessation of treatment with the one or more DMARDs, and wherein no difference in levels of the one or more biomarkers compared to the one or more reference levels is indicative of a decreased likelihood of maintaining remission following the cessation of treatment with the one or more DMARDs.
A method of treating or preventing RA in a patient undergoing treatment with one or more DMARDs, wherein:
   (i) the patient has been identified as having an increased likelihood of maintaining remission of RA following cessation of treatment with one or more DMARDs according to any method as described herein, and treatment with the one or more DMARDs is reduced or ceased; or
   (ii) the patient has been identified as having a decreased likelihood of maintaining remission of RA following cessation of treatment with one or more DMARDs according to any method described herein, and treatment with the one or more DMARDs is maintained or increased.
A therapeutic agent for use in a method of treating or preventing RA in a patient, wherein the patient has been identified as having a decreased likelihood of maintaining remission of RA following cessation of treatment with the one or more DMARDs according to any method as described herein, and the therapeutic agent is the one or more DMARDs.
An assay comprising:
   (i) purifying or labelling CD4+ T cells from a sample obtained from a patient having or suspected of having RA;
   (ii) detecting levels of one or more biomarkers in the purified or labelled CD4+ T cells; and
   (iii) comparing the levels obtained in (ii) with one or more reference levels:

wherein the levels of one or more biomarkers comprise:
(a) expression levels of at least one, two, three or more genes selected from any one of SEQ ID NOs: 1 to 19; and/or
(b) expression levels of at least one, two, three or more gene variants comprising a sequence having at least 95% homology to any one of SEQ ID Nos 1 to 19 based on nucleic acid identity over the entire length of the sequence.
A kit comprising reagents to carry out any method or assay described herein, wherein the kit comprises one or more agents capable of specifically binding to the one or more biomarkers within CD4⁺ T cells in a sample obtained from the patient.

### DETAILED DESCRIPTION OF CERTAIN EMBODIMENTS OF THE INVENTION

Certain embodiments of the present invention will now be described hereinafter, by way of example only, with reference to the accompanying drawings in which:

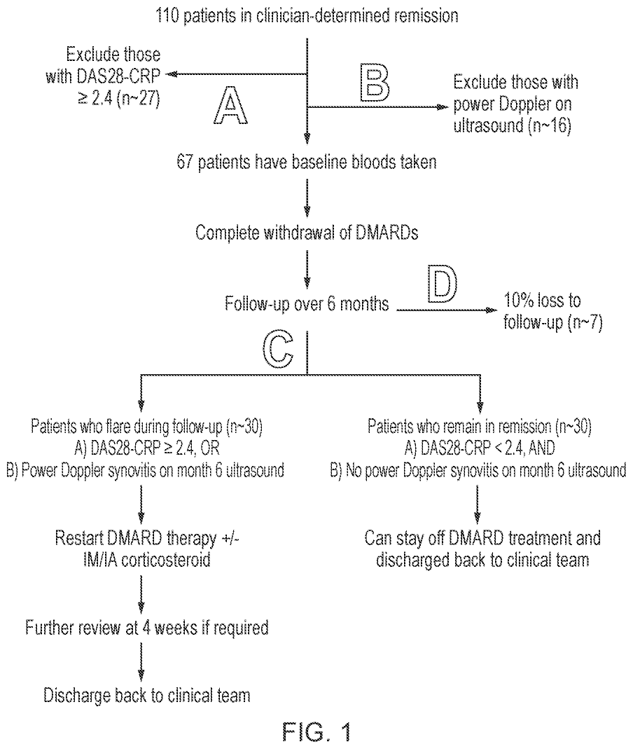

FIG. 1 shows the design of the BioRRA study together with sample size estimations. CRP, C-reactive protein; DAS, disease activity score: DMARD, disease-modifying anti-rheumatic drug; IA, intra-articular: IM, intra-muscular.

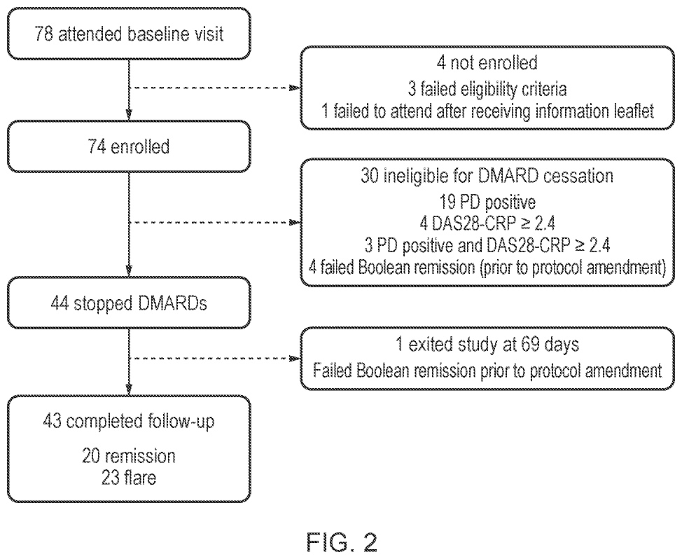

FIG. 2 shows a flow diagram with patient recruitment and outcomes.

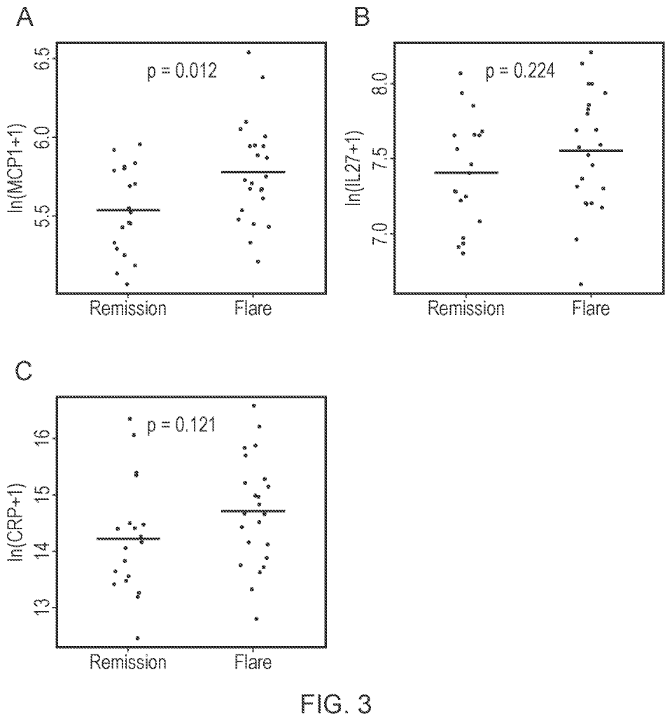

FIG. 3 shows distribution of log 2-transformed concentrations of MCP1 (A), IL-27 (B), and CRP (C) at baseline in flare and remission groups. Solid line represents mean value, statistical significance of difference in means calculated by unpaired Student's T-test.

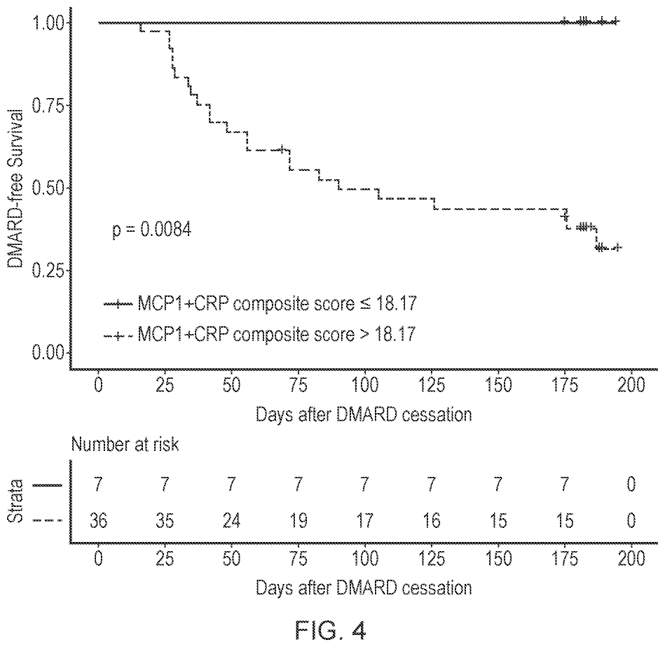

FIG. 4 shows a Kaplan-Meier plot of DMARD-free survival for the study population dichotomised by baseline composite MCP1/CRP score using the remission threshold. All patients with a negative composite score maintained DFR.

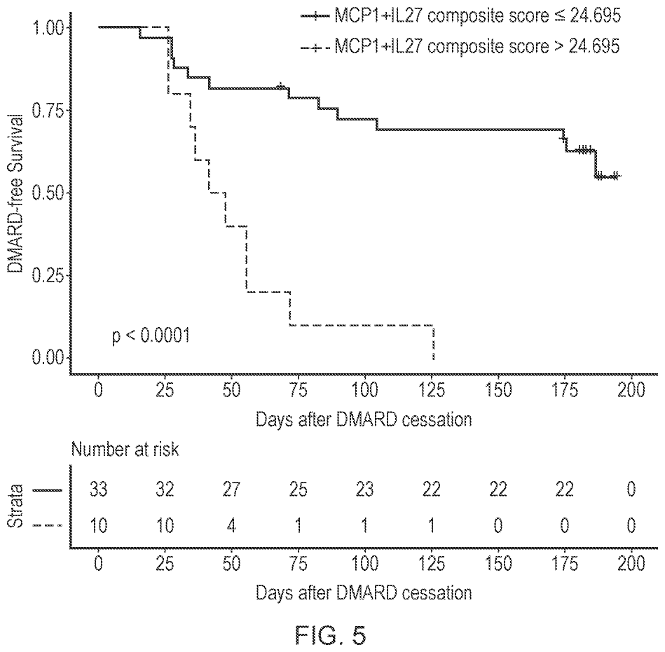

FIG. 5 shows a Kaplan-Meier plot of DMARD-free survival for the study population dichotomised by baseline composite MCP1/IL-27 score using the flare threshold. All patients with a positive composite score experienced an arthritis flare.

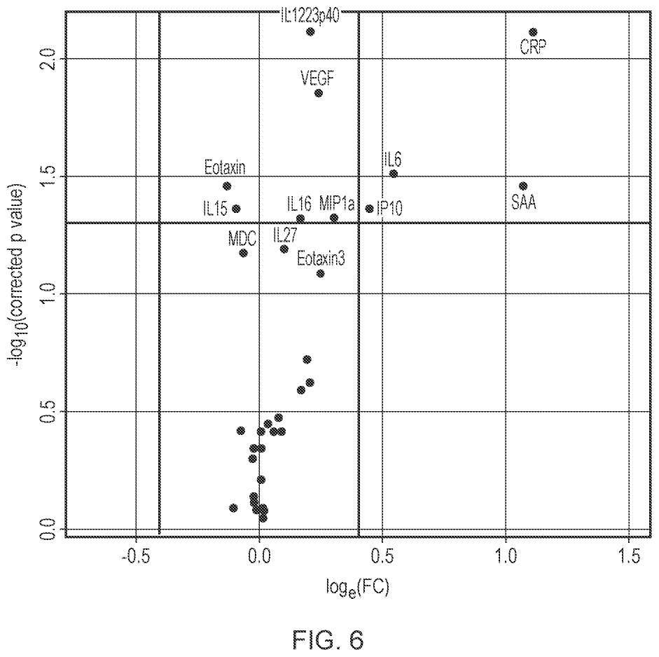

FIG. 6 shows a volcano plot of log (fold change) in chemokine/cytokine concentration between baseline versus flare visit and associated adjusted p values (Student's paired T-test, Benjamini-Hochberg) for those patients who experienced an arthritis flare. Thresholds for significance are shown at fold-change >1.5 and adjusted p<0.05. FC: fold-change.

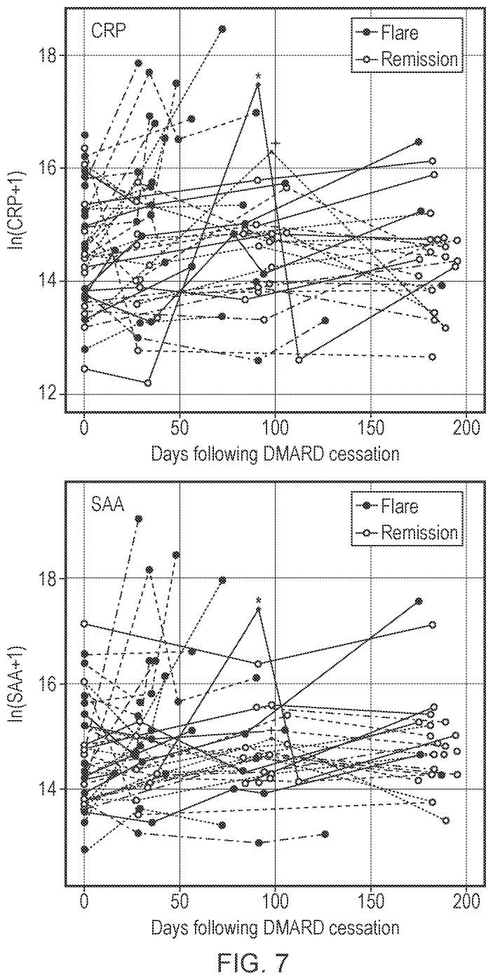

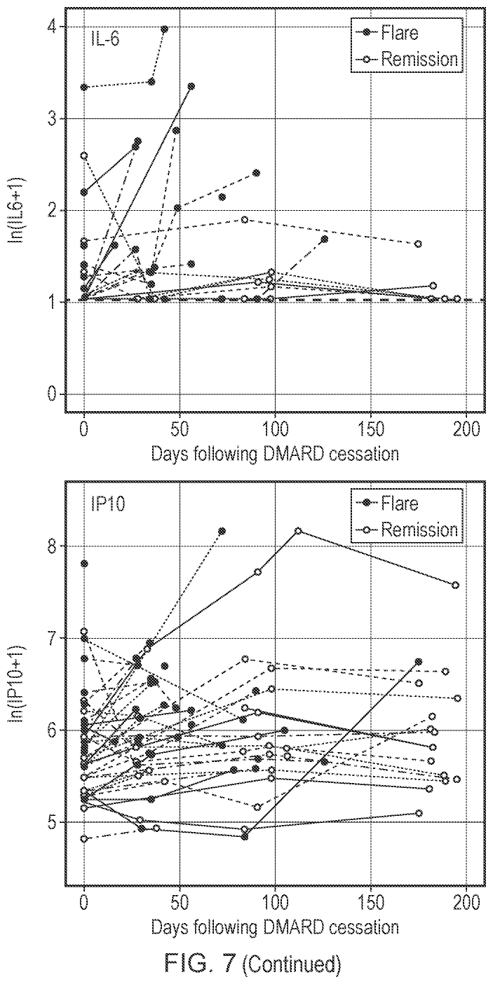

FIG. 7 shows longitudinal change in selected cytokines and chemokines. Each line represents an individual patient; those who experienced an arthritis flare are shown in red and continuous, whereas those who remained in remission are shown in grey and dashed. Spikes in CRP and SAA secondary to urinary tract infection (*) and chest infection (+) are highlighted.

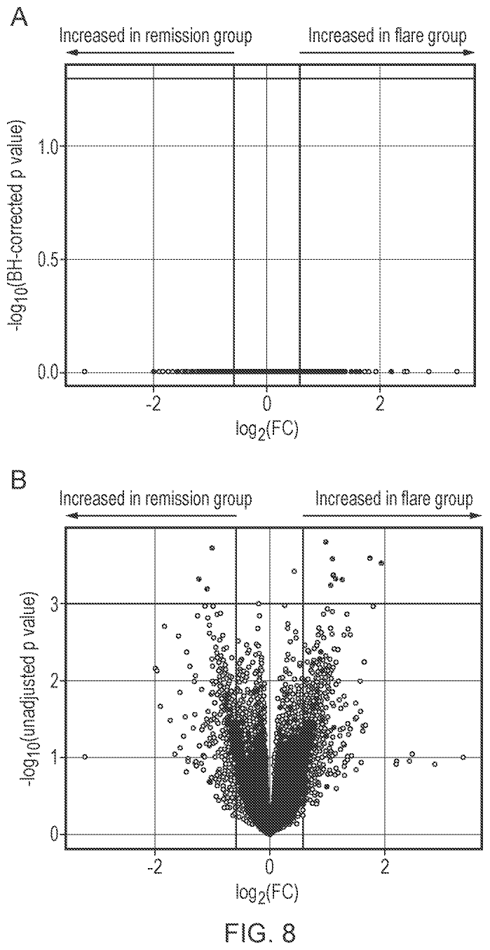

FIG. 8 shows volcano plots showing baseline differential gene expression in circulating CD4′ T cells between patients who subsequently experienced an arthritis flare versus those who remained in drug-free remission following DMARD cessation. Plots are shown with (A: adjusted p-value <0.05) and without (B: unadjusted p<0.001) multiple test correction. Horizontal lines represent log-fold change (log₂FC)>1.5.

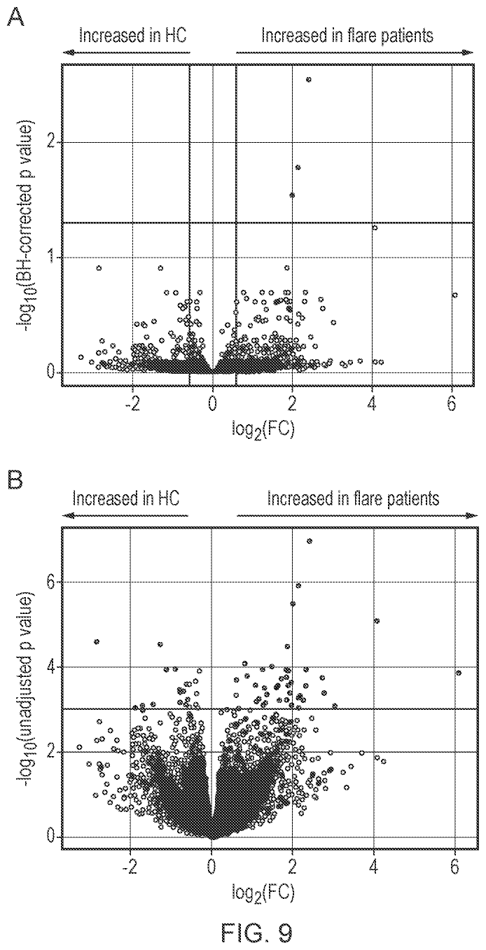

FIG. 9 shows volcano plots showing baseline differential gene expression in circulating CD4⁺ T cells between patients who subsequently experienced an arthritis flare following DMARD cessation versus healthy controls (HC). Plots are shown with (A: adjusted p-value <0.05) and without (B: unadjusted p<0.001) multiple test correction. Horizontal lines represent log-fold change (log₂FC)>1.5. Genes that exceeded both thresholds are highlighted in red.

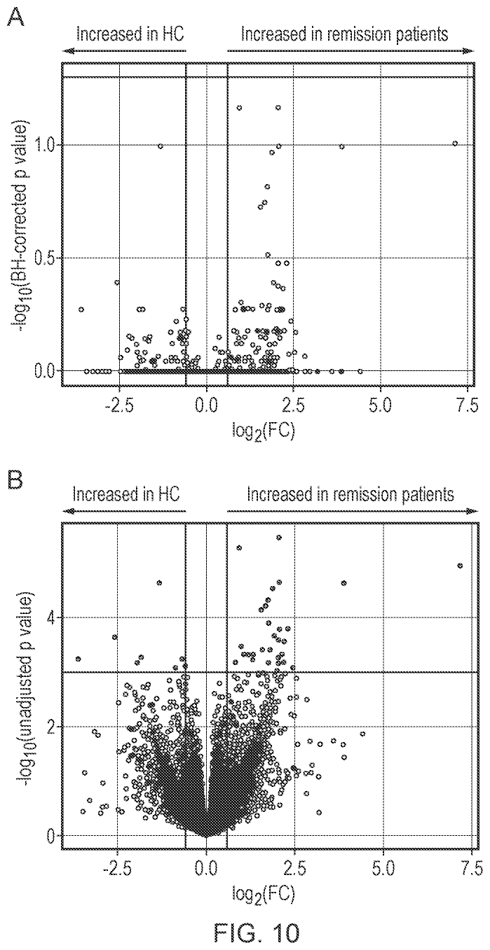

FIG. 10 shows volcano plots showing baseline differential gene expression in circulating CD4⁺ T cells between patients who subsequently remained in drug-free remission following DMARD cessation versus healthy controls (HC). Plots are shown with (A: adjusted p-value <0.05) and without (B: unadjusted p<0.001) multiple test correction. Horizontal lines represent log-fold change (log₂FC)>1.5. Genes that exceeded both thresholds are highlighted in red.

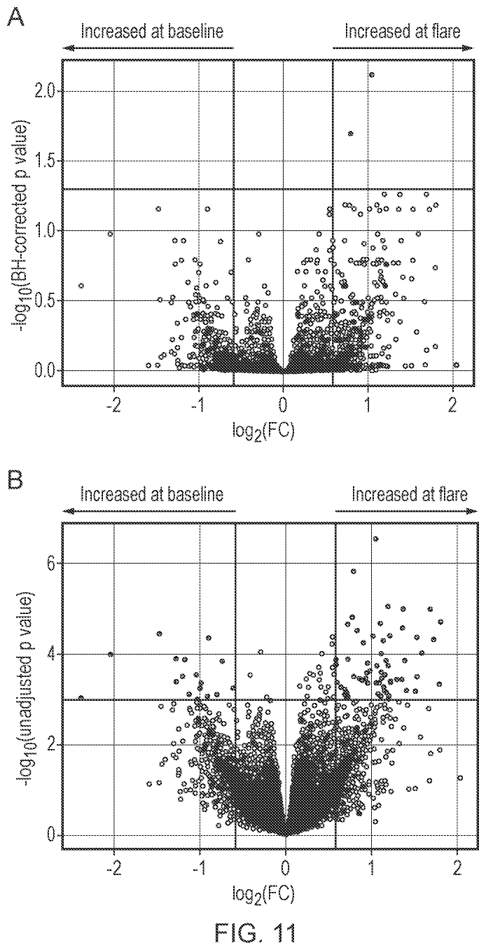

FIG. 11 shows volcano plots showing longitudinal change in gene expression between time of arthritis flare versus baseline for patients who experienced an arthritis flare following DMARD cessation. Plots are shown with (A: adjusted p-value <0.05) and without (B: unadjusted p<0.001) multiple test correction. Horizontal lines represent log-fold change (log₂FC)>1.5. Genes that exceeded both thresholds are highlighted in red.

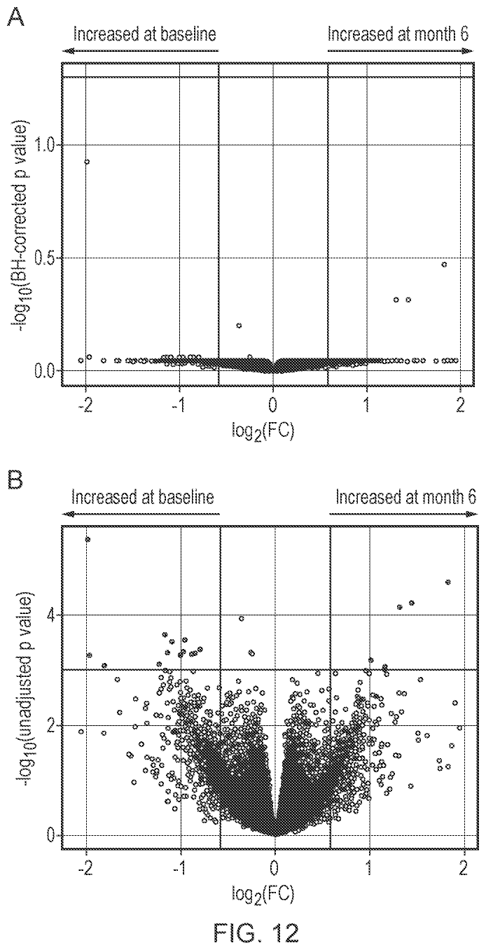

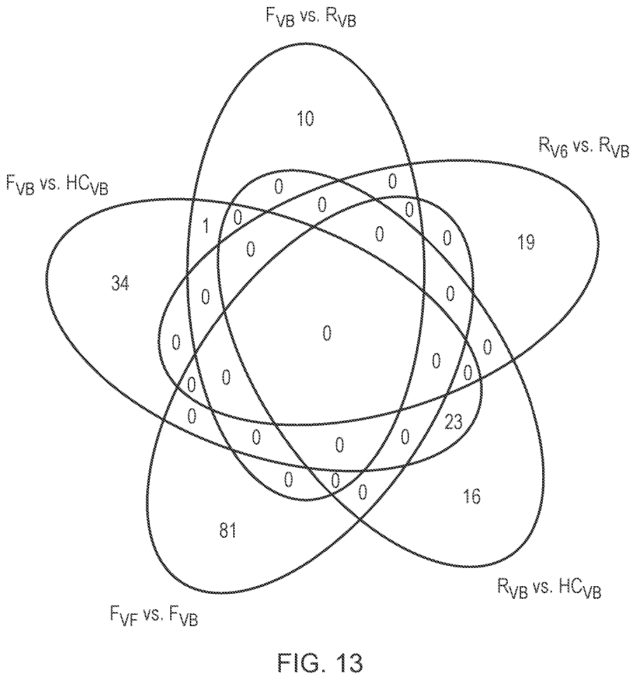

FIG. 12 shows volcano plots showing longitudinal change in gene expression between month six versus baseline for patients who remained in drug-free remission following DMARD cessation. Plots are shown with (A: FDR-corrected p-value <0.05) and without (B: unadjusted p<0.001) multiple test correction. Horizontal lines represent log-fold change (log₂FC)>1.5. Genes that exceeded both thresholds are highlighted in red.

FIG. 13 shows overlap of differentially expressed genes identified in different contrast pairs at the unadjusted p<0.001 significance threshold. F: flare patient; FV: flare visit; HC: healthy control; V6: month 6 visit; VB: baseline visit; VF: flare visit; R: remission patient.

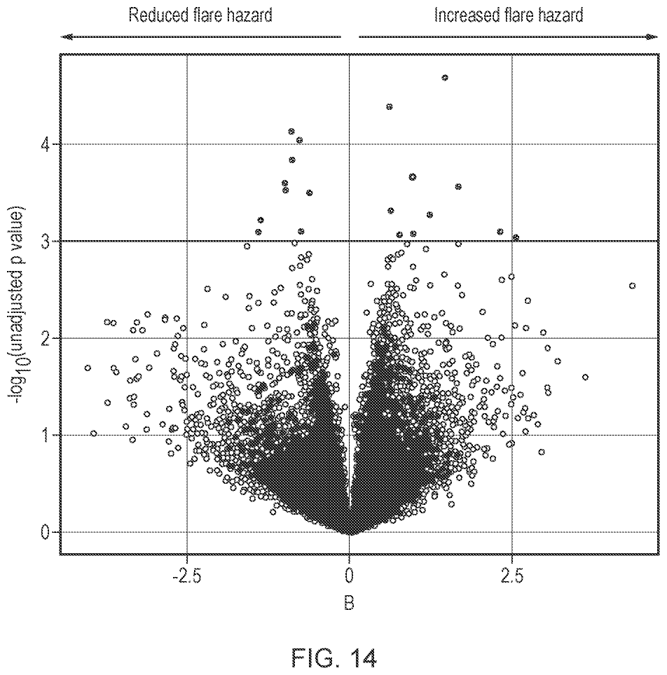

FIG. 14 shows volcano plot showing baseline gene expression in circulating CD4⁺ T cells as analysed by univariate Cox regression of time-to-flare following DMARD cessation. The horizontal line shows the unadjusted p-value <0.001 threshold—genes that exceeded this threshold are highlighted in red. B: univariate Cox regression coefficient.

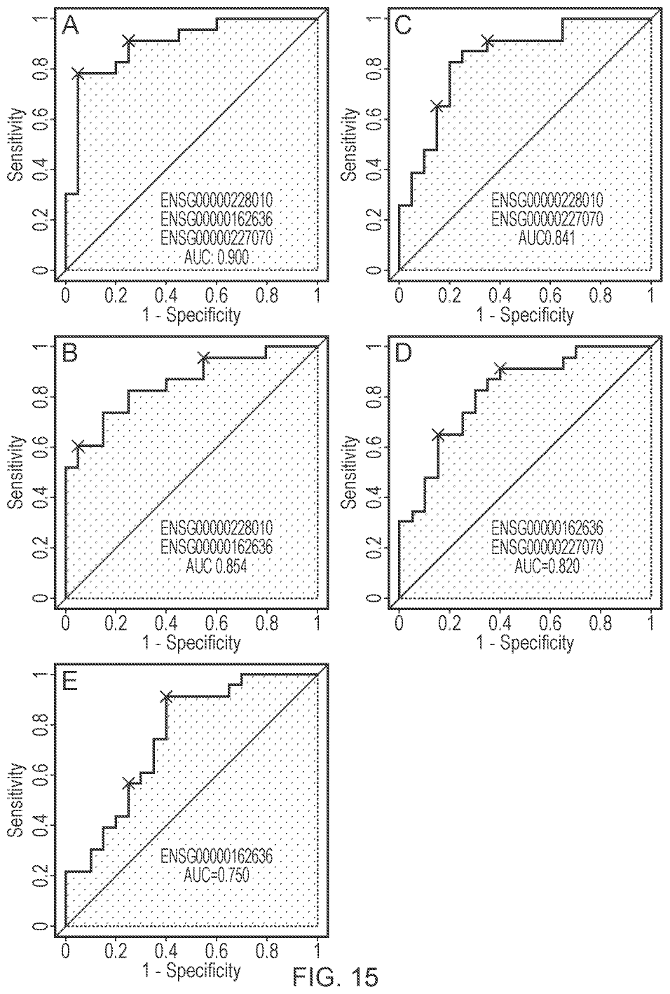

FIG. 15 shows receiver-operating characteristic curves for RNAseq composite biomarker scores for the prediction of flare following DMARD cessation. A MCP1+IL27+CRP, B: MCP1+IL27, C: MCP1+CRP, D: MCP1, E: IL27+CRP, E: CRP, F: IL27.

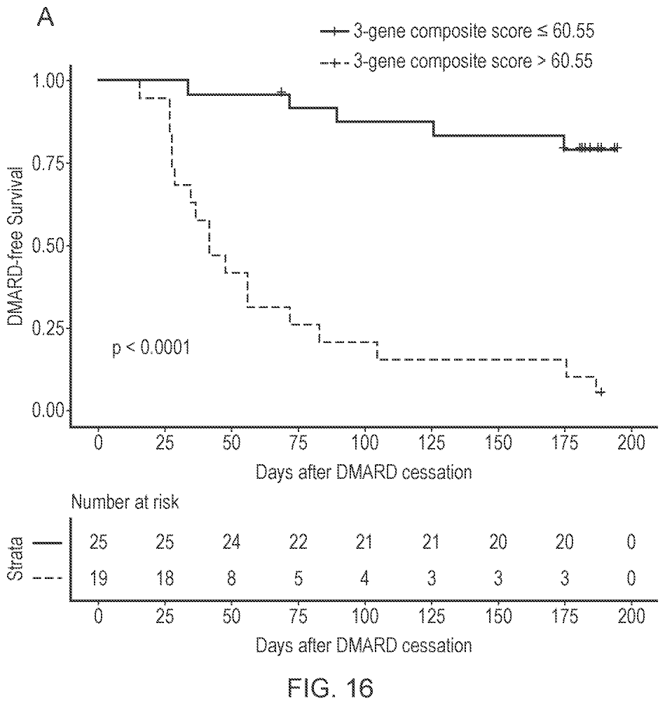

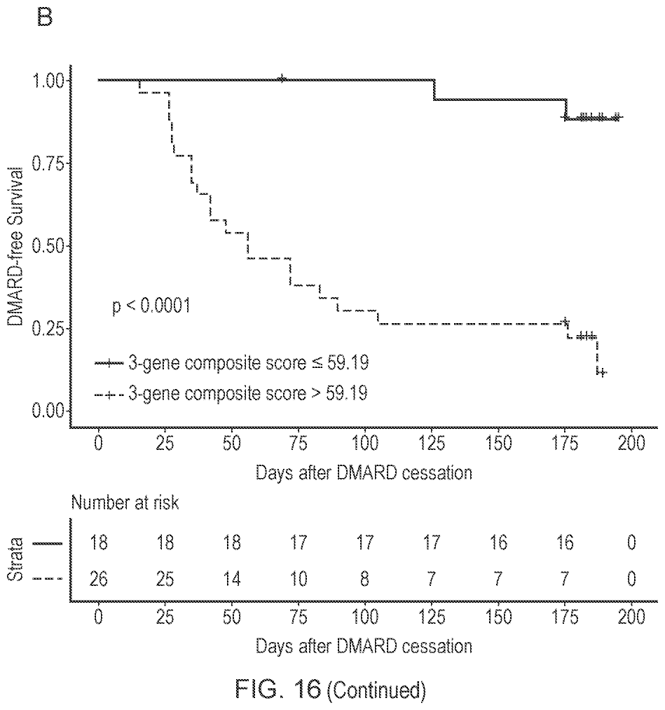

FIG. 16A: shows Kaplan-Meier plot of DMARD-free survival stratified by 3-gene composite score >60.55 (red) versus ≤60.55 (blue). B: Kaplan-Meier plot of DMARD-free survival stratified by 3-gene composite score >59.19 (red) or 559.19 (blue). P-values calculated by log-rank test.

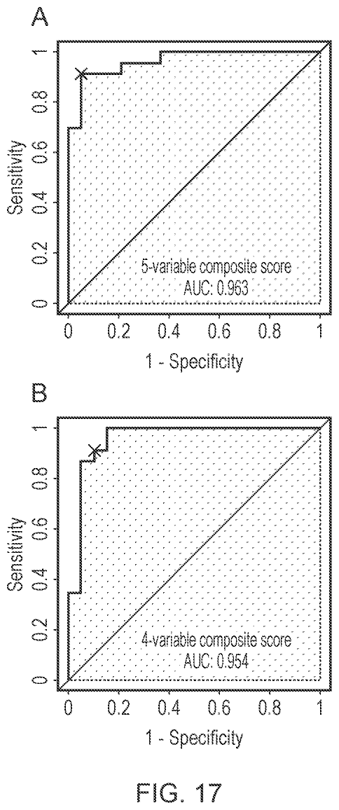

FIG. 17 shows ROC curves for 5-variable (A) and 4-variable (B—dropping ln(IL27+1)) composite scores. Threshold values used for assessment of predictive performance are highlighted by crosses.

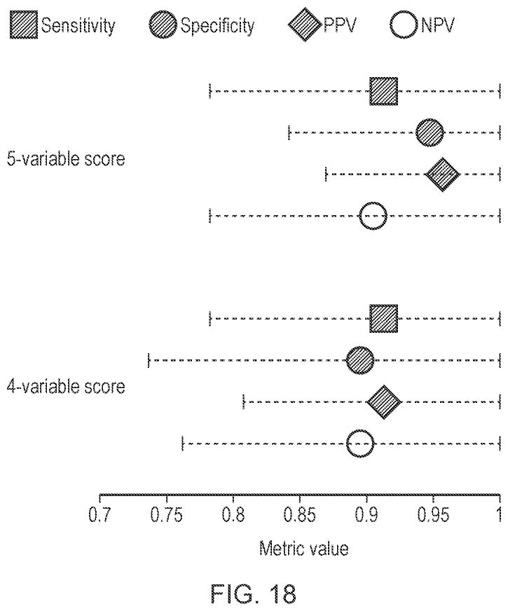

FIG. 18 shows predictive performance metrics of the two composite scores.

### SEQUENCE LISTING

SEQ ID NO: 1 shows human genomic nucleotide sequence of ENSG00000102362.

SEQ ID NO: 2 shows human genomic nucleotide sequence of ENSG00000247033.

SEQ ID NO: 3 shows human genomic nucleotide sequence of ENSG00000276571.

SEQ ID NO: 4 shows human genomic nucleotide sequence of ENSG00000204965.

SEQ ID NO: 5 shows human genomic nucleotide sequence of ENSG00000241146.

SEQ ID NO: 6 shows human genomic nucleotide sequence of ENSG00000250030.

SEQ ID NO: 7 shows human genomic nucleotide sequence of ENSG00000213296.

SEQ ID NO: 8 shows human genomic nucleotide sequence of ENSG00000229619.

SEQ ID NO: 9 shows human genomic nucleotide sequence of ENSG00000125046.

SEQ ID NO: 10 shows human genomic nucleotide sequence of ENSG00000182489.

SEQ ID NO: 11 shows human genomic nucleotide sequence of ENSG00000144366.

SEQ ID NO: 12 shows human genomic nucleotide sequence of ENSG00000237473.

SEQ ID NO: 13 shows human genomic nucleotide sequence of ENSG00000228010.

SEQ ID NO: 14 shows human genomic nucleotide sequence of ENSG00000250827.

SEQ ID NO: 15 shows human genomic nucleotide sequence of ENSG00000042286.

SEQ ID NO: 16 shows human genomic nucleotide sequence of ENSG00000231305.

SEQ ID NO: 17 shows human genomic nucleotide sequence of ENSG00000255330.

SEQ ID NO: 18 shows human genomic nucleotide sequence of ENSG00000227070.

SEQ ID NO: 19 shows human genomic nucleotide sequence of ENSG00000162636.

The practice of embodiments of the present invention employs, unless otherwise indicated, conventional techniques of chemistry, molecular biology, pharmaceutical formulation, pharmacology and medicine, which are within the skill of those working in the art.

Most general chemistry techniques can be found in Comprehensive Heterocyclic Chemistry IF (Katritzky et al., 1996, published by Pergamon Press); Comprehensive Organic Functional Group Transformations (Katritzky et al., 1995, published by Pergamon Press): Comprehensive Organic Synthesis (Trost et al., 1991, published by Pergamon); Heterocyclic Chemistry (Joule et al. published by Chapman & Hall): Protective Groups in Organic Synthesis (Greene et al., 1999, published by Wiley-Interscience); and Protecting Groups (Kocienski et al., 1994).

Most general molecular biology techniques can be found in Sambrook et al, Molecular Cloning, A Laboratory Manual (2001) Cold Harbor-Laboratory Press, Cold Spring Harbor, N.Y. or Ausubel et al., Current Protocols in Molecular Biology (1990) published by John Wiley and Sons, N.Y.

Most general pharmaceutical formulation techniques can be found in Pharmaceutical Preformulation and Formulation (2^{nd} Edition edited by Mark Gibson) and Pharmaceutical Excipients: Properties, Functionality and Applications in Research and Industry (edited by Otilia M Y Koo, published by Wiley).

Most general pharmacological techniques can be found in A Textbook of Clinical Pharmacology and Therapeutics (5^{th} Edition published by Arnold Hodder).

Most general techniques on the prescribing, dispensing and administering of medicines can be found in the British National Formulary 72 (published jointly by BMJ Publishing Group Ltd and Royal Pharmaceutical Society).

Unless defined otherwise, all technical and scientific terms used herein have the same meaning as commonly understood by one of ordinary skill in the art to which this disclosure belongs. For example, the Concise Dictionary of Biomedicine and Molecular Biology. Juo, Pei-Show, 2^{nd} ed., 2002, CRC Press; The Dictionary of Cell and Molecular Biology, 3^{rd} ed., Academic Press; and the Oxford University Press, provide a person skilled in the art with a general dictionary of many of the terms used in this disclosure. For chemical terms, the skilled person may refer to the International Union of Pure and Applied Chemistry (IUPAC).

Units, prefixes and symbols are denoted in their Système International d'Unités (SI) accepted form. Numeric ranges are inclusive of the numbers defining the range.

Biomarkers of Drug-Free Remission of RA

The present invention relates to biomarkers of drug-free remission of RA. In certain embodiments, the invention provides a method of determining the likelihood of a patient suffering or who has suffered from RA maintaining remission following cessation of treatment with at least one or more DMARDs.

The patient may be any individual. Typically, the patient is human. Typically, the patient has RA or has suffered from RA but is in remission following treatment with one or more DMARDs. For example, the patient may no longer exhibit any symptoms of RA. In other words, the patient may be asymptomatic. Alternatively, the patient may exhibit reduced or dramatically reduced symptoms of RA.

Currently “remission of RA” is typically defined in terms of disease activity. In 2011, the ACR and EULAR published joint remission criteria to develop a definition of remission in RA that is “stringent but achievable”. The remission criteria is outlined in the Table below:

| | |
|---|---|
| Boolean | At any time point, patient must satisfy the following: |
| definition | Tender joint count ≤1 |
| | Swollen joint count ≤1 |
| | C reactive protein ≤1 mg/dL (10 mg/L) |
| | Patient global assessment ≤1 (on a 0-10 scale) |
| Index | At any time point, patient must have a Simplified |
| definition | Disease Activity Index score of ≤3.3 |

The American College of Rheumatology (ACR)/European League Against Rheumatism (EULAR) 2011 remission criteria for rheumatoid arthritis. Reproduced from Annals of the Rheumatic Diseases, Felson et al., volume 70, pages 404-413, copyright 2011

In certain embodiments, the method comprises determining the patient's ACR/EULAR Boolean Remission criteria (i.e., definition) as Tender joint count ≤1, Swollen joint count ≤1, patient global assessment of ≤1 (i.e., less than or equal to 1 on a 0-10 clinical scale) and C reactive protein ≤1 mg/dL (10 mg/L). In particular, patients determined as having Tender joint count ≤1 (i.e., less than or equal to 1 on a 0-10 clinical scale), Swollen joint counts ≤1 (i.e., less than or equal to 1 on a 0-10 clinical scale), patient global assessment of ≤1 (i.e., less than or equal to 1 on a 0-10 clinical scale), and C reactive protein ≤1 mg/dL (10 mg/L) may be classified as in remission for RA. Alternatively, patients determined as having Tender joint count >1 (i.e., more than 1 on a 0-10 clinical scale), Swollen joint count >1 (i.e., more than 1 on a 0-10 clinical scale), patient global assessment of ≤1 (i.e., less than or equal to 1 on a 0-10 clinical scale), and/or C reactive protein >1 mg/dL (10 mg/L) may be classified as having active RA (i.e., not in remission).

In certain embodiments, the method comprises determining whether the patient has achieved DAS28-CRP remission, as extensively used in current clinical practice and outlined in the Table below:

| DAS28-CRP remission | [0.56√(TJC28) + 0.28√(SJC28) + |
|---|---|
| (Fleischmann et al., 2015) | 0.36ln(CRP + 1) + 0.014(VASₚₐₜᵢₑₙₜ) + |
| | 0.96] < 2.4 |

The final clinical remission definition used in the BioRRA study. CRP, C-reactive protein in mg/L; DAS28, disease activity score in 28 joints; SJC28, swollen joint count in 28 joints; TJC28, tender joint count in 28 joints; VASₚₐₜᵢₑₙₜ, patient visual analogue score in millimetres (on 0-100 mm scale).

In certain embodiments, the method comprises determining the patients DAS28-CRP criteria (i.e. definition) as ≤2.4. For example, patients having DAS28-CRP criteria as <2.4 (i.e., less than 2.4) may be classified as in remission for RA. Alternatively, patients having DAS28-CRP criteria as ≥2.4 (i.e., more than or equal to 2.4) may be classified as having active RA (i.e., not in remission).

The patient may have been treated with any one or more DMARDs. In certain embodiments, the patient may have been treated with a single DMARD (i.e., a monotherapy). In certain embodiments, the patient may have been treated with more than one DMARD (i.e., a combination therapy).

In certain embodiments, the patient may have been treated with one or more bDMARDs such as etanercept, infliximab, adalimumab, certolizumab, golimumab, rituximab, abatacept, tocilizumab and/or sarilumab, In certain embodiments, the patient may have been treated with one or more targeted synthetic DMARDs (tsDMARD). For example, the patient may have been treated with a tsDMARD selected from tofacitinib, baricitinib, filgotinib, upadicitinib and/or any other drug in this class.

In certain embodiments, the patient may have been treated with one or more conventional synthetic DMARDs. In certain embodiments, the patient may have been treated with one or more of methotrexate, leflunomide, sulfasalazine and/or hydroxychloroquine. For example, the patient may have been treated with methotrexate only (i.e., methotrexate monotherapy). Alternatively, the patient may have been treated with methotrexate in combination with an additional DMARD (i.e., methotrexate combination therapy). In certain embodiments, the patient may have been treated with combinations of DMARDs that don't include methotrexate.

For example, the patient may have been treated with sulfasalazine in combination with hydroxychloroquine.

In the methods of the invention, the levels of one or more biomarkers in a sample of CD4⁺ T cells obtained from the patient are detected. Typically, the sample of CD4⁺ T cells comprises purified CD4⁺ T cells (i.e., isolated from other cell types) or labelled CD4⁺ T cells (to allow the specific detection of biomarkers within the CD4+ T cells).

The sample may be from any tissue or bodily fluid from the patient containing CD4⁺ T cells. Typically, the sample is derived from a blood sample. The sample is typically processed before the method is carried out, for example protein, RNA or DNA extraction may be carried out. The biomarkers in the sample of CD4⁺ T cells (i.e., polynucleotide or protein) may be cleaved either physically or chemically, for example using a suitable enzyme. In one embodiment, polynucleotides in the sample are copied or amplified, for example using a PCR based method. Methods for obtaining samples from patients are well known in the art.

In certain embodiments, levels of one or more biomarkers in purified CD4⁺ T cells are detected. In other words, the CD4⁺ T cells in the sample are isolated or substantially isolated from other cell types prior to detecting levels of the one or more biomarkers. Any method of purifying CD4⁺ T cells may be used. For example, CD4⁺ T cells may be isolated from plasma or serum samples following established protocols in the art (see, for example, Pratt et al., 2011). In some embodiments, CD4⁺ T cells may be isolated following column-based immunoprecipitation methods. The levels of biomarkers in the purified CD4⁺ T cells may then be measured using any one or more techniques as discussed herein.

The phrase “detecting levels of one or more biomarkers” typically means detecting the expression levels (i.e., amount) of one or more genes (i.e., polynucleotide sequences) in the CD4⁺ T cells. In certain embodiments, the level of a transcription product (i.e., mRNA, tRNA or micro RNA) or its complement (i.e., a cDNA complement of the transcription product) are detected in the sample. In certain embodiments, the level of a translation product (i.e., protein or polypeptide sequences or metabolite thereof) are detected in the sample. Gene expression can be measured by detecting the presence, quantity, or activity of a DNA, RNA, or polypeptide, or modifications thereof (e.g., splicing, phosphorylation, and acetylation) associated with a given gene.

Detecting levels of one or more biomarkers may comprise contacting a polynucleotide or protein in a sample from the patient with a specific binding agent for the biomarker and determining whether the agent binds to the polynucleotide or protein, wherein binding of the agent to the biomarker can be used to quantify levels of the biomarker.

A specific binding agent is an agent that binds with preferential or high affinity to the biomarker but does not bind or binds with only low affinity to other polynucleotides or proteins. The specific binding agent may be a probe or primer. The probe may be a protein (such as an antibody as described below) or an oligonucleotide. The probe may be labelled or may be capable of being labelled indirectly. The binding of the probe to the polynucleotide or protein may be used to immobilise either the probe or the polynucleotide or protein. The primer may be a PCR primer.

In some embodiments, expression levels of one or more genes are detected by microarray. In some embodiments, expression levels of one or more genes are detected by PCR techniques such as quantitative reverse transcriptase polymerase chain reaction (qRT-PCR). Procedures for performing qRT-PCR are well known in the art. In some embodiments, the expression levels of one or more genes are detected by sequencing methods such as next generation sequencing.

In some embodiments, the specific binding agent may be capable of specifically binding the amino acid sequence encoded by any one or more of the gene sequences described herein (i.e., encoded by any one or more of SEQ ID Nos 1 to 19). For example, the specific binding agent may be an antibody or antibody fragment. In some embodiments, methods of detecting levels of one or more biomarkers comprise measuring the levels of proteins present in the sample. This may be achieved by methods known to those skilled in the art. Such methods include immunoassays, for example immunohistochemistry, ELISA, Western blots, immunoprecipitation followed by SDS-PAGE and immunocytochemistry, and the like.

In certain embodiments, levels of one or more biomarkers in labelled CD4⁺ T cells are detected. In other words, CD4⁺ T cells may be labelled in a sample that includes other cell types, in order to allow the specific detection of biomarkers within the CD4⁺ T cells. For example, the methods may comprise identifying and/or measuring CD4⁺ T cell surface protein markers in combination with the levels of one or more biomarkers within the CD4⁺ T cells. In some embodiments, the PrimeFlow™ RNA assay (Affymetrix eBisocience Ltd.) is used to detect levels of one or more biomarkers within a labelled sample of CD4⁺ T cells. This technique uses intracellular in situ-hybridisation of fluorescent probes to target genes in a system which is compatible with existing flow-cytometry equipment (Affymetrix eBioscience, 2017). Such techniques allow the measurement of cell-specific gene expression without the need for cell subset isolation or RNA extraction.

In certain embodiments, the levels of one or more biomarkers in a sample of CD4 T cells comprise expression levels of at least 1, 2, 3, 4, 5, 6, 7, 8, 9, 10, 11, 12, 13, 14, 15, 16, 17, 18, 19 or more genes selected from the differentially expressed genes identified in Example 4 below.

For example, the levels of one or more biomarkers may comprise expression levels of at least 1, 2, 3, 4, 5, 6, 7, 8, 9, 10 or more genes selected from any one of SEQ ID Nos 1 to 19. In certain embodiments, expression levels of at least 3 or more of any one of SEQ ID Nos 1 to 19 are detected. Typically, the expression levels of at least 1, 2, 3, 4, 5 or more of any one of SEQ ID Nos 13, 19, 18, 4, 8, 2, 9, 5, 3, 14 and 15 are detected. More typically, the expression levels of at least SEQ ID Nos 18, 19 and 13 are detected.

In certain embodiments, expression levels of at least one, two, three or more gene variants comprising a sequence having at least 95%, 96%, 97%, 98%, 99% or more homology to any one of SEQ ID Nos 1 to 19 based on nucleic acid identity over the entire length of the sequence are detected. For example, expression levels of at least 1, 2, 3, 4, 5 or more gene variants comprising a sequence having at least 95% homology to any one of SEQ ID Nos 13, 19, 18, 4, 8, 2, 9, 5, 3, 14 or 15 based on nucleic acid identity over the entire length of the sequence may be detected. Typically, the expression levels of one or more gene variants comprising a sequence having at least 95% homology to SEQ ID Nos 18, 19 or 13 based on nucleic acid identity over the entire length of the sequence are detected.

The above mentioned homology is calculated on the basis of nucleic acid identity (sometimes referred to as “hard homology”). The UWGCG Package provides programs including GAP, BESTFIT, COMPARE, ALIGN and PILEUP that can be used to calculate homology or line up sequences (for example used on their default settings). The BLAST algorithm can also be used to compare or line up two sequences, typically on its default settings. Software for performing a BLAST comparison of two sequences is publicly available through the National Center for Biotechnology Information (http://www.ncbi.nlm nih.gov/). This algorithm is further described below. Similar publicly available tools for the alignment and comparison of sequences may be found on the European Bioinformatics Institute website (http://www.ebi.ac.uk), for example the ALIGN and CLUSTALW programs.

In certain embodiments, detecting levels of one or more biomarkers in a sample of CD4⁺ T cells obtained from a patient comprise outputting, optionally on a computer, (i) an indication of the levels of the one or more biomarkers and (ii) that this indicates whether or not the patient is likely to maintain remission following the cessation of treatment with the one or more DMARDs.

In certain embodiments, the levels of one or more biomarkers in a sample of CD4⁺ T cells obtained from the patient are compared with one or more reference (i.e., control) levels. For example, reference levels may be obtained by detecting levels of one or more biomarkers in a sample of CD4⁺ T cells from healthy individuals or patients known to have maintained remission of RA following cessation of treatment with one or more DMARDs. In addition or alternatively, reference levels may be obtained by detecting levels of one or more biomarkers in a sample of CD4⁺ T cells from patients known to have shown flare or relapse of RA symptoms following cessation of treatment with the one or more DMARDs.

In certain embodiments, a difference in levels of the one or more biomarkers compared to the one or more reference levels is indicative of an increased likelihood of maintaining remission following the cessation of treatment with one or more DMARDs. In other words, a difference in levels of the one or more biomarkers compared to the one or more reference levels indicates that the patient is likely not to exhibit any onset or flare of RA symptoms upon reduction or withdrawal of any DMARD therapy. In such embodiments, DMARD therapy to the patient may be reduced or ceased unless the patient exhibits any onset or flare of RA symptoms.

In certain embodiments, no difference in levels of the one or more biomarkers compared to the one or more reference levels is indicative of a decreased likelihood of maintaining remission following the cessation of treatment with one or more DMARDs. In other words, no difference in levels of the one or more biomarkers compared to the one or more reference levels indicates that the patient is likely to exhibit an onset or flare of RA symptoms upon reduction or withdrawal of any DMARD therapy. In such embodiments, DMARD therapy to the patient may be re-commenced or increased.

The phrase “a difference in levels” is understood to mean either a decrease or an increase in levels of the one or more biomarkers compared to the one or more reference levels.

In some embodiments, the levels of the one or more biomarkers are decreased as compared to the one or more reference levels. For example, levels of the one or more biomarkers may be decreased about 1.1 fold, about 1.2 fold, about 1.3 fold, about 1.4 fold, about 1.5 fold, about 2 fold, about 3 fold, about 4 fold, about 5 fold or more as compared to the one or more reference levels. In other words, the one or more biomarkers may be about 90% or less, about 80% or less, about 75% or less, about 70% or less, about 65% or less, about 50% or less, about 33% or less, about 25% or less, about 20% or less as compared to the one or more reference levels.

In preferred embodiments, levels of the one or more biomarkers are increased as compared to the one or more reference levels. For example, the level of the one or more biomarkers may be increased about 1.1 fold, about 1.2 fold, about 1.3 fold, about 1.4 fold, about 1.5 fold, about 2 fold, about 3 fold, about 4 fold, about 5 fold or more as compared to the one or more reference levels. In other words, the one or more biomarkers may be more than about 110%, more than about 120%, more than about 130%, more than about 140%, more than about 150%, more than about 200%, more than about 300%, more than about 400%, more than about 500% or more as compared to the one or more reference levels.

In certain embodiments, detecting levels of one or more biomarkers may further comprise detecting levels of one or more autoantibodies in a sample obtained from a patient. For example, methods may further comprise detecting levels of anti-citrullinated protein antibodies (ACPA) and/or rheumatoid factor (RF) in a sample obtained from the patient and comparing the levels with one or more reference levels, wherein an increase in levels of ACPA and/or RF is indicative of a decreased likelihood of maintaining remission following the cessation of treatment with one or more DMARDs.

Detection of Cytokines

In any method described herein, the method further comprises (i) detecting levels of at least one or more cytokines in a sample obtained from the patient, and (ii) comparing the levels obtained in (i) with one or more reference levels. Typically, a difference in levels of the one or more cytokines compared to the one or more reference levels is indicative of an increased likelihood of maintaining remission following the cessation of treatment with one or more DMARDs. Alternatively, no difference in levels of the one or more biomarkers compared to the one or more reference levels is indicative of a decreased likelihood of maintaining remission following the cessation of treatment with the one or more DMARDs.

The levels of one or more cytokines may be detected by any method known in the art. Typically, one or more cytokines in a sample from the patient are contacted with a specific binding agent for the cytokine and any binding of the agent to the cytokine determined. For example, binding of the agent to the cytokine may be used to quantify levels of the cytokine.

In certain embodiments, the agent may be an antibody or antibody fragment. For example, methods may comprise measuring the levels of the cytokine protein present in the sample. This may be achieved by any methods known in the art.

The sample obtained from the patient used to detect the levels of one or more cytokine may be any sample containing cytokines as described herein. For example, the sample may comprise labelled or purified CD4⁺ T cells. Alternatively, a further sample (such as serum or plasma) may be obtained from the patient.

In certain embodiments, the method further comprises comparing the levels of the one or more cytokines with one or more reference (i.e., control) levels. Typically, the reference levels are obtained by detecting levels of one or more cytokines from healthy individuals or patients already known to have maintained remission of RA following cessation of treatment with the one or more DMARDs. In some embodiments, the reference levels are obtained by detecting levels of one or more cytokines from patients known to have shown flare or relapse of RA symptoms following cessation of treatment with the one or more DMARDs.

In certain embodiments, a difference in levels of the one or more cytokines compared to the one or more reference levels is indicative of an increased likelihood of maintaining remission following the cessation of treatment with one or more DMARDs. In other words, a difference in levels of the one or more cytokines compared to the one or more reference levels is indicative that the patient is likely not to exhibit any onset or flare of RA symptoms upon reduction or withdrawal of DMARD therapy. In such embodiments, DMARD therapy to the patient may be reduced or ceased unless the patient exhibits any onset or flare of RA symptoms.

In certain embodiments, no difference in levels of the one or more cytokines compared to the one or more reference levels is indicative of a decreased likelihood of maintaining remission following the cessation of treatment with one or more DMARDs. In other words, no difference in levels of the one or more cytokines compared to the one or more reference levels indicates that the patient is likely to exhibit an onset or flare of RA symptoms upon reduction or withdrawal of any DMARD therapy. In such embodiments, DMARD therapy to the patient may be re-commenced or increased.

The phrase “a difference in levels” is understood to mean either a decrease or an increase in levels of the one or more cytokines compared to the one or more reference levels. In certain embodiments, the levels of the one or more cytokines are decreased as compared to the one or more reference levels. For example, the level of the one or more cytokines may be decreased by about 1.1 fold, about 1.2 fold, about 1.3 fold, about 1.4 fold, about 1.5 fold, about 2 fold, about 3 fold, about 4 fold, about 5 fold or more as compared to the one or more reference levels. In other words, the level of the one or more cytokines may be less than about 90%, less than about 80%, less than about 75%, less than about 70%, less than about 65%, less than about 50%, less than about 33%, less than about 25%, less than about 20% or less as compared to the one or more reference levels.

In certain embodiments, the levels of the one or more cytokines are increased as compared to the one or more reference levels. For example, the level of the one or more cytokines may be increased by about 1.1 fold, about 1.2 fold, about 1.3 fold, about 1.4 fold, about 1.5 fold, about 2 fold, about 3 fold, about 4 fold, about 5 fold or more as compared to the one or more reference levels. In other words, the level of the one or more cytokines may be more than about 110%, more than about 120%, more than about 130%, more than about 140%, more than about 150%, more than about 200%, more than about 300%, more than about 400%, more than about 500% or more as compared to the one or more reference levels.

In certain embodiments, at least 1, 2, 3, 4, 5 or more cytokines are detected. In the methods of the invention, any cytokine predictive of drug-free remission in RA may be detected. Typically, the one or more cytokines comprise at least 1, 2, 3, 4, 5 or more of interleukin-27 (IL-27), CRP, serum amyloid A (SAA), Interferon gamma-induced protein 10 (IP-10), IL-6, monocyte chemoattractant protein-1 (MCP-1), MIP 1-α, VEGF, IL-12. IL-23 and/or IL-16. For example, the one or more cytokines may comprise at least 1, 2, 3, 4, 5 or all 6 of IL-27, CRP, SAA, IP-10, IL-6 and/or MCP-1.

In preferred embodiments, expression levels of SEQ ID Nos 18, 19 and 13 or variants thereof are detected in combination with any cytokine predictive of drug-free remission in RA such as those listed above. For example, expression levels of SEQ ID Nos 18, 19 and 13 may be detected in combination with at least 1, 2, 3, 4, 5 or more of interleukin-27 (IL-27), CRP, serum amyloid A (SAA), Interferon gamma-induced protein 10 (IP-10), IL-6, and/or monocyte chemoattractant protein-1 (MCP-1). Such combinations are unexpectedly and particularly effective at determining the likelihood of a patient maintaining remission of RA following cessation of treatment with at least one or more DMARDs.

Clinical Criteria

In any method described herein, the method may further comprise determining baseline clinical variables predictive of drug-free remission in RA. Typically, the method further comprises determining the patients ACR/EULAR Boolean Remission criteria as described herein. For example, in certain embodiments the invention relates to the unexpected finding that patients having ACR/EULAR Boolean remission at baseline is associated with a significantly lower risk of arthritis flare following DMARD cessation.

In certain embodiments, the method further comprises determining the patients tender joint count, global assessment on a 0-10 clinical scale, swollen joint count and C reactive protein levels. Patients determined as having ACR/EULAR Boolean Remission criteria (i.e., definition) as Tender joint count ≤1, global assessment of <1, swollen joint count ≤1 and C reactive protein ≤1 mg/dL (10 mg/L) is indicative that the patient has an increased likelihood of maintaining remission of RA following cessation of treatment with one or more DMARDs.

Alternatively, patients determined as having Tender joint count >1 (i.e., more than 1 on a 0-10 clinical scale), global assessment of >1 (i.e., more than 1 on a 0-10 clinical scale), swollen joint count >1 (i.e., more than 1 on a 0-10 clinical scale) and/or C reactive protein >1 mg/dL (10 mg/L) is indicative that the patient has a decreased likelihood of maintaining remission following the cessation of treatment with one or more DMARDs.

In certain embodiments, the method may further comprise determining the patients DAS28-CRP as described herein. For example, patients having DAS28-CRP criteria as less than 2.4 is indicative that the patient has an increased likelihood of maintaining remission of RA following cessation of treatment with one or more DMARDs. Alternatively, patients having DAS28-CRP criteria as more than or equal to 2.4 is indicative that the patient has a decreased likelihood of maintaining remission following the cessation of treatment with one or more DMARDs.

Methods of Treatment

Also provided herein are methods of treating or preventing RA in a patient undergoing treatment with one or more DMARDs. Therapy and prevention includes, but is not limited to, preventing, alleviating, reducing, curing or at least partially arresting symptoms and/or complications resulting from or associated with RA. The patient can have a genetic predisposition to RA. The patient may be in remission for RA and/or can be asymptomatic.

In certain embodiments, the patient is (or has been) identified as having an increased likelihood of maintaining remission of RA following cessation of treatment with one or more DMARDs according to any method as described herein. In such embodiments, treatment with the one or more DMARDs is reduced (or tapered) or ceased (i.e., stopped). Alternatively, the patient is (or has been) identified as having a decreased likelihood of maintaining remission of RA following cessation of treatment with the one or more DMARD according to any method as described herein. In such embodiments, treatment with the one or more DMARDs is maintained (i.e., continued at the same level or dosage) or increased (i.e., increased to a higher level or dosage).

Non-limiting routes of administration of the one or more DMARDs include by oral, intravenous, intraperitoneal, subcutaneous, intramuscular, topical, intradermal, intranasal or intrabronchial administration.

One or more DMARDs may be administered in an amount effective to prevent, inhibit or delay the development of RA. Suitable doses and dosage regimes for a given patient and one or more DMARDs can be determined using a variety of different methods, such as body-surface area or body-weight, or in accordance with specialist literature and/or individual hospital protocols. Doses may be further adjusted following consideration of a patient's neutrophil count, renal and hepatic function, and history of any previous adverse effects to the one or more DMARDs. Doses may also differ depending on whether a DMARD is used alone or in combination with another therapy. The other therapy may be a specific treatment directed at the disease or condition suffered by the patient or directed at a symptom of such a disease or condition. For example, the other therapy may be a general therapy aimed at treating or improving the condition of a patient with inflammation. For example, treatment with glucocorticoids, salicylates, nonsteroidal anti-inflammatory drugs (NSAIDs), analgesics, aminosalicylates and/or corticosteroids may be combined with one or more other DMARDs.

Such therapies may be administered separately or sequentially to a patient as part of the same therapeutic regimen. The skilled person will recognize that further modes of administration, dosages of one or more DMARDs and treatment regimens can be determined by the treating physician according to methods known in the art.

In certain embodiments, the patient has ceased therapy with one or more DMARDs prior to detecting levels of one or more biomarkers in a sample of CD4 T cells obtained from the patient. If the patient is identified as having a decreased likelihood of maintaining remission of RA following cessation of treatment with the one or more DMARDs, the patient is ideally treated with the one or more DMARDs as soon as possible to minimize the chances of flare or onset of RA symptoms. Thus, in some embodiments the method is for preventing the symptoms of RA or decreasing the risk of symptoms of RA re-occurring in the patient. Typically, a patient is treated immediately or shortly after being identified as having a decreased likelihood of maintaining remission of RA following cessation of treatment with one or more DMARDs.

In certain embodiments, the patient is identified as having an increased likelihood of maintaining remission of RA following cessation of treatment with the one or more DMARDs. In such embodiments, treatment with the one or more DMARDs may be ceased. Typically, the method of the invention may be repeated at intervals (i.e., every 3 months) after the cessation of DMARD therapy, and DMARD therapy re-commenced if the biomarkers suggest that is the optimal course of action. Thus, the invention further provides:
a method of determining the likelihood of a patient who has ceased treatment with one or more DMARDs maintaining remission of RA, the method comprising:
   (i) detecting levels of one or more biomarkers in a sample of CD4⁺ T cells obtained from the patient; and
   (ii) comparing the levels obtained in (i) with one or more reference levels;

wherein a difference in levels of the one or more biomarkers compared to the one or more reference levels is indicative that treatment with the one or more DMARDs should continue to be ceased, and wherein no difference in levels of the one or more biomarkers compared to the one or more reference levels is indicative that treatment with the one or more DMARDs should be re-commenced.

In some embodiments, the levels of one or more biomarkers are detected in the patient every 1, 2, 3, 4, 5 or 6 or more months after ceasing treatment with the one or more DMARDs. Typically, the levels of one or more biomarkers are detected for as long as the patient remains in remission.

Assays

The invention also provides assays comprising purifying or labelling CD4⁺ T cells from a sample obtained from a patient having or suspected of having RA. Any method may be used to purify CD4⁺ T cells from a sample. For example, CD4⁺ T cells may be isolated following column-based immunoprecipitation as discussed above. Any method may be used to label CD4⁺ T cells from a sample, as discussed above.

In the assays of the invention, levels of one or more biomarkers comprise expression levels of at least 1, 2, 3, 4, 5, 6, 7, 8, 9, 10 or more genes selected from any one or more of SEQ ID Nos 1 to 19. Typically, expression levels of at least 3 or more of any one of SEQ ID Nos 1 to 19 are detected. More typically, expression levels of at least 1, 2, 3, 4, 5 or more of any one of SEQ ID Nos 13, 19, 18, 4, 8, 2, 9, 5, 3, 14 and 15 are detected. In preferred embodiments, the expression levels of at least SEQ ID Nos 18, 19 and 13 are detected.

In certain embodiments, expression levels of at least one, two, three or more gene variants comprising a sequence having at least 95%, 96%, 97%, 98%, 99% or more homology to any one of SEQ ID Nos 1 to 19 based on nucleic acid identity over the entire length of the sequence are detected. For example, expression levels of at least 1, 2, 3, 4, 5 or more gene variants comprising a sequence having at least 95% homology to any one of SEQ ID Nos 13, 19, 18, 4, 8, 2, 9, 5, 3, 14 or 15 based on nucleic acid identity over the entire length of the sequence may be detected. In preferred embodiments, the expression levels of one or more gene variants comprising a sequence having at least 95% homology to SEQ ID Nos 18, 19 or 13 based on nucleic acid identity over the entire length of the sequence are detected.

In certain embodiments, the assay further comprises detecting levels of at least one or more cytokines as described herein. For example, the assay may comprise detecting expression levels of SEQ ID Nos 18, 19 and 13 in combination with one or more cytokine predictive of drug-free remission in RA. Typically, the one or more cytokines comprise at least 1, 2, 3, 4, 5 or more of interleukin-27 (IL-27), CRP, serum amyloid A (SAA), Interferon gamma-induced protein 10 (IP-10), IL-6, monocyte chemoattractant protein-1 (MCP-1), MIP 1-α, VEGF, IL-12. IL-23 and/or IL-16. For example, the one or more cytokines may comprise IL-27, CRP, SAA, IP-10, IL-6 and/or MCP-1.

In certain embodiments, the assay further comprises determining baseline clinical variables that are predictive of drug-free remission in RA. Typically, the assay may further comprise determining the patients ACR/EULAR Boolean Remission criteria as discussed above.

In certain embodiments, levels of one or more biomarkers in the CD4⁺ T cells are compared with one or more reference levels as described herein. Typically, the one or more biomarkers in the CD4 T cells are increased by at least about 1.1 fold, about 1.2 fold, about 1.3 fold, about 1.4 fold, about 1.5 fold, about 2 fold, about 3 fold, about 4 fold, about 5 fold or more as compared to the one or more reference levels.

Kits

The invention also provides kits comprising reagents to carry out any of the methods or assays as described herein.

The kits of the invention comprise one or more agents capable of specifically binding to the one or more biomarkers in a sample of CD4⁺ T cells obtained from the patient. In some embodiments, the one or more agents are labelled. For example, an agent may comprise a detection moiety and/or a binding moiety specific for the one or more biomarkers. In some embodiments, the agent comprises an enzyme as a detection moiety, and the kit further comprises a substrate of the enzyme.

In some embodiments, the one or more agents are primers or probes that may be used in the detection of the one or more biomarkers described herein. These probes and primers may be used for determining the likelihood of a patient maintaining remission of RA following cessation of treatment with one or more DMARDs. Polynucleotide sequences disclosed herein may also be used in the design of primers or probes to detect the one or more biomarkers.

Such primers, probes and other polynucleotide fragments will preferably be at least 10, preferably at least 15 or at least 20, for example at least 25, at least 30 or at least 40 nucleotides in length. They will typically be up to 40, 50, 60, 70, 100 or 150 nucleotides in length. Probes and fragments can be longer than 150 nucleotides in length, for example up to 200, 300, 400, 500, 600, 700 nucleotides in length, or even up to a few nucleotides, such as five or ten nucleotides, short of a full-length polynucleotide sequence disclosed herein.

Primers and probes for detecting the one or more biomarkers may be designed using any suitable design software known in the art. For example, primers and probes may be designed using regions of any one of the sequence set forth in SEQ ID NOs: 1 to 19. Homologues of these polynucleotide sequences would also be suitable for designing primers and probes.

Such homologues typically have at least 95%, at least 97% or at least 99% homology, for example over a region of at least 15, 20, 30, 100 more contiguous nucleotides. The homology may be calculated on the basis of nucleotide identity (sometimes referred to as “hard homology”). Methods for determining homology are described herein.

The primers or probes may be present in an isolated or substantially purified form. They may be mixed with carriers or diluents that will not interfere with their intended use and still be regarded as substantially isolated. They may also be in a substantially purified form, in which case they will generally comprise at least 90%, e.g. at least 95%, at least 98% or at least 99%, of polynucleotides of the kit.

In some embodiments, the kit comprises one or more agents capable of specifically binding to any one of SEQ ID Nos 1 to 19. For example, the kit may comprise one or more agents capable of specifically binding to at least SEQ ID NO: 18, 19 and 13.

In some embodiments, the kit further comprises instructions for using the kit to predict the likelihood of a patient maintaining remission of rheumatoid arthritis (RA) following cessation of treatment with one or more disease-modifying anti-rheumatic drugs (DMARDs).

In some embodiments, the kit may further comprise one or more additional components such as reagents and/or apparatus necessary for carrying out an in vito assay, e.g. buffers, fixatives, wash solutions, blocking reagents, diluents, chromogens, enzymes, substrates, test tubes, plates, pipettes etc.

The kit of certain embodiments of the invention may advantageously be used for carrying out a method of certain embodiments of the invention and could be employed in a variety of applications, for example in the diagnostic field or as a research tool. It will be appreciated that the parts of the kit may be packaged individually in vials or in combination in containers or multi-container units. Typically, manufacture of the kit follows standard procedures which are known to the person skilled in the art.

### Example 1—Study Design

The inclusion, exclusion and DMARD-cessation criteria for the Biomarkers of Remission in Rheumatoid Arthritis (BioRRA) Study are summarised in the Table below:

| | | |
|---|---|---|
| Inclusion | 1. | Clinical diagnosis of rheumatoid arthritis made by |
| criteria | | consultant rheumatologist at least 12 months previously |
| | 2. | Current single or combination use of methotrexate, |
| | | sulfasalazine and/or hydroxychloroquine |
| | 3. | Arthritis currently in remission, as judged clinically by |
| | | referring healthcare professional |
| | 4. | Willing to consider DMARD withdrawal |
| Exclusion | 1. | Use of biologic therapy within the past 6 months |
| criteria | 2. | Received steroids within past 3 months (enteral, |
| | | parenteral or intra-articular) |
| | 3. | Use of any DMARD other than methotrexate, |
| | | sulfasalazine or hydroxychloroquine within the past 6 |
| | | months (or past 12 months for leflunomide) |
| | 4. | Current pregnancy, or pregnancy planned within next 6 |
| | | months |
| | 5. | Current participation within another clinical trial |
| | 6. | Inability to provide informed consent |
| DMARD- | 1. | Clinical remission, defined as DAS28-CRP < 2.4 * |
| cessation | | |
| criteria | | |

Inclusion, exclusion and DMARD cessation criteria for the BioRRA study. Patients who met the inclusion and exclusion criteria were enrolled for the baseline visit, but only those patients who met the DMARD-cessation criteria stopped DMARDs and continued in the study follow-up: ACR/EULAR Boolean remission was initially used as the clinical remission definition, though this was subsequently changed to DAS28-CRP < 2.4

Inclusion and Remission Criteria

Patients were recruited to the study if they had a clinical diagnosis of RA, with fulfilment of diagnostic criteria assessed retrospectively by the research team after study enrolment.

The composite scoring system of DAS28-CRP was used to define clinical remission in the present study, as extensively used in current clinical practice (Table below):

| DAS28-CRP remission | [0.56√(TJC28) + 0.28√(SJC28) + |
|---|---|
| (Fleischmann et al., 2015) | 0.36ln(CRP + 1) + 0.014(VASₚₐₜᵢₑₙₜ) + |
| | 0.96] < 2.4 |

The final clinical remission definition used in the BioRRA study. CRP, C-reactive protein in mg/L; DAS28, disease activity score in 28 joints; SJC28, swollen joint count in 28 joints; TJC28, tender joint count in 28 joints; VASₚₐₜᵢₑₙₜ, patient visual analogue score in millimetres (on 0-100 mm scale).

The final clinical remission definition used in the BioRRA study. CRP, C-reactive protein in mg/L; DAS28, disease activity score in 28 joints; SJC28, swollen joint count in 28 joints; TJC28, tender joint count in 28 joints; VASₚₐₜᵢₑₙₜₛ, patient visual analogue score in millimetres (on 0-100 mm scale).

The use of CRP was used owing to its specificity for inflammation. A threshold of DAS28-CRP<2.4 was used as the definition of clinical remission—this is lower than the remission threshold for DAS28-ESR (<2.6) and is in line with recently published data that recommends this lower remission threshold (Fleischmann et al., 2015). Values of CRP below the detectable threshold of the local clinical laboratory (<5 mg/L) were recorded as zero for the purposes of DAS28-CRP calculation.

Study Design and Sample Size Estimation

The design of the study together with sample size estimations are summarised in FIG. 1.

Patients who satisfied both DAS28-CRP<2.4 and an absence of PD signal on ultrasound assessment (see below) at the baseline visit were eligible for DMARD cessation. Further administrations of methotrexate, sulfasalazine and/or hydroxychloroquine were immediately stopped without tapering. Non-DMARDs, including folic acid, remain unaltered and non-steroidal anti-inflammatory drug (NSAID) use was permitted. Research blood tests including serum, plasma, peripheral blood mononuclear cells (PBMCs), CD4⁺ T cell isolation and whole blood RNA TEMPUS™ tubes (Applied Biosystems, Foster City, Calif., USA) were collected at baseline, month 3 and month 6 following DMARD cessation. An US scan was performed again at the month 6 visit. If a patient experienced a flare of their arthritis (defined as DAS28≥2.4) or if they developed PD synovitis on the month 6 scan, then they could receive rescue glucocorticoids and were discharged from the study with their DMARD therapy promptly recommenced by their referring rheumatologist. Those patients who remained in remission at 6 months with no PD synovitis were discharged back to their referring rheumatologist without DMARD therapy. 74 patients were recruited to the study, of which 44 stopped DMARDs.

Prospective Clinical Variable Assessment

Pre-specified clinical variables were recorded prospectively at the baseline study visit, as listed in the Table below:

| Baseline variable | Data type |
|---|---|
| Age | Continuous |
| Sex | Binary |
| 28 tender joint count | Discrete |
| 28 swollen joint count | Discrete |
| Patient arthritis visual analogue score (range 0-100) | Continuous |
| ESR | Continuous |
| CRP | Continuous |
| RhF positive | Binary |
| ACPA positive | Binary |
| DAS28-CRP | Continuous |
| DAS28-ESR | Continuous |
| Fulfilment of ACR/EULAR Boolean remission | Binary |
| HAQ-DI (range 0-3) | Continuous |
| Patient global health score (range 0-100) | Continuous |
| Patient pain score (range 0-100) | Continuous |

Baseline clinical variables recorded prospectively in the BioRRA study. CRP: C-reactive protein; ESR: erythrocyte sedimentation rate; RhF: rheumatoid factor; ACPA: anti-citrullinated peptide antibody; HAQ-DI

Included in these variables is the Health Assessment Questionnaire Disability Index (HAQ-DI), a self-completed questionnaire that quantifies physical disability that has been extensively validated in the setting of RA and other chronic diseases.

Retrospective Clinical Variable Assessment

Data were obtained for a range of pre-specified variables, as listed in the Table below. These data were obtained both by patient interview at baseline visit, and from clinical notes review. Historical clinical records were available for all patients and were assessed by a single reviewer. It was acknowledged that the most reliable source of information might differ depending on the variable of interest. A systematic methodology of recording clinical variables was therefore implemented, as shown in the Table below:

| Variable | Data type | Data source |
|---|---|---|
| Year of RA diagnosis | Continuous | Medical notes |
| Months from symptom onset | Continuous | Medical notes |
| to first rheumatology clinic | | |
| review | | |
| Months from first | Continuous | Medical notes |
| rheumatology clinic review | | |
| to commencement of first | | |
| DMARD | | |
| Months since last change in | Continuous | Medical notes |
| DMARD therapy (dose | | |
| and/or drug) | | |
| Months since last | Continuous | Most recent of either |
| glucocorticoid | | medical notes or patient |
| | | interview |
| Smoking status | Categorical | Patient interview |
| | (current/ | |
| | previous/never) | |
| Weekly alcohol unit intake | Continuous | Patient interview |
| Methotrexate use | Categorical | Medical notes |
| | (current/ | |
| | previous/never) | |
| Sulfasalazine use | Categorical | Medical notes |
| | (current/ | |
| | previous/never) | |
| Hydroxychloroquine use | Categorical | Medical notes |
| | (current/ | |
| | previous/never) | |
| Other previous DMARDs | Free text | Medical notes |

Baseline clinical variables recorded retrospectively in the BioRRA study

Assessment of RA Criteria

Patients were recruited on the grounds of a clinical diagnosis of RA made by a consultant rheumatologist, with satisfaction of formal classification criteria assessed retrospectively after patient recruitment.

Healthy Control Participants

In order to provide a control group for subsequent transcriptomic analyses, four healthy participants were recruited. Each participant donated blood at four time points to mirror those of the patient participants—i.e. baseline, month 1, month 3 and month 6. Healthy participants donated blood between 9 am and 1 pm where possible to minimise circadian variation in laboratory samples. Blood was left to stand at room temperature to mimic the equivalent period of transit for patient samples, before being processed for CD4⁺ T cell extraction and subsequent downstream RNA/DNA applications using identical protocols to those of the patient participants.

CD4⁺ T Cell Isolation

CD4⁺ T cells were isolated from 27 ml of whole blood drawn into three 9 ml ethylenediaminetetraacetic acid (EDTA) tubes following the protocol of Pratt (2011). Briefly, monocytes were first depleted by the use of an anti-CD36/anti-glycophorim A cross-linking reagent (Rosettesep human monocyte depletion cocktail) that crosslinks monocytes to erythrocytes forming immunorosettes, which were separated by centrifugation after the addition of the erythrocyte aggregation agent HetaSep®. The supernatant was then collected and the remaining CD4⁺ T cells were positively selected by automated anti-CD4 antibody-based magnetic isolation (Easisep® whole blood CD4⁺ selection kit and Robosep® automated cell separator). By the prior removal of CD4′° expressing monocytes this extraction method is able to obtain CD4⁺ T cell purities of over 98%, compared to approximately 90% for single step column-based immunoprecipitation methods (Pratt et al., 2012). After cell counting (see below), 2×10⁵ cells were transferred to a single well of a 96-well V-bottom plate for flow cytometry processing (see below).

CD4⁺ T Cell Lysis

Purified CD4⁺ T cell isolates were transferred to 30 ml universal tube and washed by addition of cold (4° C.) calcium- and magnesium-free Hanks balanced salt solution (HBSS)+1% foetal calf serum (FCS) to a total volume of 25 ml. The tube was then centrifuged at 400 g for 7 minutes at 4° C., after which the supernatant was removed by aspiration using a vacuum-driven glass pipette. In an RNase-free open workbench area, the pellet was then resuspended in cold (4° C.) Qiagen Buffer RLT Plus+1% β-mercaptoethanol dependent on the number of T cells to be lysed; if <5 million cells then 350 μL of Buffer/β-mercaptoethanol was added, whereas if 25 million cells then 600 μL of Buffer/β-mercaptoethanol was added (in line with the manufacturer's protocol). The suspension was thoroughly mixed by pipetting and vortexing before transfer to a QIA-shredder column. Lysis of the T cells was completed by centrifugation of the column at 13,000 g for 2 minutes, and the lysate was then stored at −80° C.

Peripheral Blood Mononuclear Cell Isolation

PBMCs were isolated from 18 ml of whole blood drawn into two 9 ml EDTA tubes. The blood was diluted in HBSS+2 mM EDTA to a total volume of 40 ml. The diluted blood was then split to two 20 ml volumes that were layered by slow pipetting above 15 ml of Lymphoprep® within a 50 ml centrifuge tube. The layered tubes were then centrifuged at 895 g for 30 minutes with minimal acceleration and deceleration speeds to maintain layering of the sample. After centrifugation, PBMCs were removed from the interface layer using a Pasteur pipette and transferred to a fresh 50 ml centrifuge tube. The PBMCs were then immediately washed in cold (4° C.) HBSS+1% FCS to a total volume of 50 ml and centrifuged at 600 g for 7 minutes at 4° C. to remove any residual Lymphoprep®. The supernatant was discarded, and the pellet resuspended in 50 ml of cold (4° C.) HBSS+1% FCS before centrifuging at 250 g for 7 minutes at 4° C. to remove any platelets. The supernatant was discarded, and the pellet resuspended in 7 ml of cold (4° C.) HBSS+1% FCS and strained through a 70 μm nylon filter to a new 50 ml centrifuge tube (to exclude clumped cells), which was kept on ice for immediate cell counting. After cell counting, 2×10⁵ cells were transferred to a well of a 96-well V-bottom plate and stored at 4° C.

Serum Separation

Blood drawn in to a single 8 ml serum separator clot activator tube was centrifuged at 1,800 g for 12 minutes. The supernatant (i.e. serum) was then carefully removed by pipetting and divided to 1 ml aliquots, and frozen at −80° C.

Cell Counting

PBMCs and CD4⁺ T cells were counted within 10 μL of diluted sample placed underneath a cover slip mounted upon a Bürker haemocytometer chamber. The number of cells within 25 squares was then manually counted using alight microscope. The total number of cells present in the sample was calculated as per the Formula below:

calculation of cell number by Bürker haemocytometer chamber counting: Total cells=[volume (ml)]×[dilution factor]×[number of cells in 25 squares]×10⁴

CD4⁺ Purity Check by Flow Cytometry

Unfixed samples of PBMCs and CD4⁺ T cells stored in 96-well V-bottom plates at 4° C. were stained and analysed by flow cytometry within 18 hours of sample isolation. Firstly, the plate was centrifuged at 400 g for 3 minutes and the supernatant removed by flicking. Cells were then resuspended in 50 μL of flow cytometry antibody mixture by thorough pipetting and incubated in the dark for 30 minutes at 4° C. After this, 100 μL of flow cytometry buffer was added to each well and the plate then centrifuged at 400 g for 3 minutes. After removal of the supernatant by flicking, the stained cells were then resuspended in 150 μL of flow cytometry buffer and the plate centrifuged at 400 g for 3 minutes. After removal of the supernatant by flicking, the cells were resuspended in 200 μL of flow cytometry buffer and transferred to individual cytometry tubes. Flow cytometry data was recorded using a FACSCanto-II cytometer and FACSDiva software (Becton Dickinson Biosciences, San Jose, Calif., USA). Analysis of flow cytometry data was performed using FlowJo software (FlowJo LLC Data Analysis Software, Ashland, Oreg., USA). PBMC samples were first gated on side-scatter area (SSC-A) and width (SSC-W) to identify singlets, which were then gated on SSC-A and forward-scatter area (FSC-A) to exclude debris. The resulting population was then gated on compensated CD3 and compensated CD4 to identify CD3⁺CD4⁺ T cells, SSC-A and compensated CD14 to identify CD14⁺ monocytes, and SSC-A and compensated CD19 to identify CD19′ B-cells. These gates were then applied to the CD4⁺ T cell isolate matched to the individual patient where available to assess purity.

CD4⁺ T Cell RNA Extraction

Frozen CD4⁺ T cell lysates (see above) were thawed at room temperature in an RNase-free open workbench area. Thawed lysates were mixed by pipetting and transferred to RNase-free conical-bottom 2 ml microcentrifuge tubes. In order to remove residual magnetic nanoparticles remaining from the CD4⁺ T cell isolation procedure, the lysates were centrifuged at maximum speed (13000 rpm) for 2 minutes. The supernatant was then carefully removed using a P1000 pipette (taking care not to disturb the pellet of magnetic nanoparticles at the bottom of the tube) and transferred to a Qiagen AllPrep DNA Mini spin column placed within a 2 ml collection tube.

The sample was then centrifuged at maximum speed (13000 rpm) for 30 seconds

The collection tube containing the flow-through from the DNA column was then processed according to the manufacturer's instructions, including on-column DNase digestion and all steps designated as ‘optional’ according to the manufacturer's instructions. Final purified RNA was eluted from the RNeasy Mini spin column using a volume of RNase-free water determined by total number of CD4⁺ T cells present in the initial sample (see above) as follows: 30 μL for <5×10⁶ cells, or 40 μL for 25×10⁸ cells. The amount of RNA present in each sample was then quantified using a NanoDrop™ ND1000 ultraviolet (UV) spectrophotometer (Thermo Fisher Scientific), with 2 μg of each sample (or the total sample if <2 μg) stored at −80° C. for RNA sequencing processing (see below).

Next-Generation RNA Sequencing (RNAseq)

The quantity and estimated RNA integrity (RINe) of RNA samples was measured by gel electrophoresis using a Tapestation™ 4200 machine (Agilent). Following quantification, 1.5 μg of total RNA per sample was used for RNAseq processing; where total RNA<1.5 μg, the entire sample was used. Total RNA was processed using the TruSeq™ Stranded mRNA Library Prep Kit (Illumina), according to the ‘High Sample Protocol’ section of the manufacturer's instructions. First, messenger RNA (mRNA) was enriched from the purified total RNA by poly-A selection using poly-T oligo attached magnetic beads in two rounds of purification. Enriched mRNA was then fragmented by heating with magnesium cations, and then incubated with reverse transcriptase to synthesise first strand cDNA for each sample. This step was performed in the presence of Actinomycin D to prevent DNA-dependent synthesis of a second strand. The mRNA was then degraded with RNase and second strand cDNA was then synthesised by incubation with DNA Polymerase I. Double-stranded cDNA was then isolated using solid phase reversible immobilisation (SPRI) paramagnetic beads (Agencourt™ AMPure™ XP beads, Beckman Coulter Genomics), after which the 3′ ends were adenylated to facilitate sequencing adaptor binding. The Illumina sequencing adaptors contained three key functional elements: an amplification element required for amplification of the cDNA by polymerase chain reaction, a sequencing element required for the sequencing reaction, and a unique index element to allow identification of each individual patient sample. cDNA that had successfully ligated with adaptors was selectively amplified by poymerase chain reaction. Amplified cDNA was then diluted to equimolar concentrations and pooled before sequencing.

RNA sequencing was performed using an Illumina NextSeg™ 500 in high-output mode. This configuration delivered 400 million reads over 75 cycles for 40 samples loaded across 4 lanes per flow cell. Sequencing was performed in batches across 4 separate flow cell sequencing runs. Samples were allocated to sequencing batches such that computational correction for any batch-to-batch variation at the level of either the RNA extraction (6 batches) or RNA sequencing (4 batches) could be achieved, according to a predetermined experimental design using the duplicate correlation command of the ‘limma’ Bioconductor/R package (v3.32.5) (Ritchie et al., 2015). Using this approach, it was possible to sequence all 136 CD4⁺ T cell samples to a depth of 10 million reads per sample, with 75 bp single-end reads.

Mutiplex Cytokne/Chemokine Electrochemiluminescence Assays

Previously separated serum samples stored at −80° C. (see above) were thawed at 37° C., mixed by vortexing and then centrifuged for 2 minutes at maximum speed (13000 rpm) to separate any contaminating debris. Serum was then transferred to 96-well V-PLEX™ plates (MesoScale Discovery) and processed according to the manufacturer's instructions. The volumes of sample loaded per well, together with the fold dilution and dilution method for each plate is detailed in the Table below, as specified by the manufacturer:

| | Sample | | |
|---|---|---|---|
| | volume | Fold | |
| V-PLEX ™ plate | (μL) | dilution | Dilution method |
| Cytokine panel 1 (human) | 25 | 2 | In-plate |
| Chemokine panel 1 | 12.5 | 4 | In-plate |
| (human) | | | |
| Proinflammatory panel 1 | 25 | 2 | In-plate |
| (human) | | | |
| Th17 panel 1 (human) | 12.5 | 4 | In-plate |
| Vascular injury panel 2 | 10 | 1000 | Three serial 10-fold |
| (human) | | | dilutions prior to |
| | | | addition to plate |

Volume, fold dilution and dilution method for samples according to V-PLEX ™ plate.

The assays included on each plate are detailed in the Table below:

| V-PLEX ™ plate | Assays | |
|---|---|---|
| Cytokine panel 1 (human) | GM-CSF | IL-15 |
| | IL-1α | IL-16 |
| | IL-5 | IL-17A |
| | IL-7 | TNF-β |
| | IL-12/23 p40 subunit | VEGF-A |
| Chemokine panel 1 (human) | Eotaxin (CCL11) | MlP-1α (CCL3) |
| | MIP-1β (CCL4) | IL-8(HA) |
| | Eotaxin-3 (CCL26) | MCP-1 (CCL2) |
| | TARC (CCL17) | MDC (CCL22) |
| | IP-10 (CXCL10) | MCP-4 (CCL13) |
| Proinflammatory panel 1 | IFN-γ | IL-8 |
| (human) | IL-1β | IL-10 |
| | IL-2 | IL-12p70 subunit |
| | IL-4 | IL-13 |
| | IL-6 | TNF-α |
| Th17 panel 1 (human) | IL-17A | IL-27 |
| | IL-21 | IL-31 |
| | IL-22 | MIP-3α (CCL20) |
| | IL-23 | |
| Vascular Injury panel 2 | SAA | VCAM-1 |
| (human) | hsCRP | ICAM-1 |

Cytokine and chemokine assays by V-PLEX ™ plate type. CCL: C-C motif chemokine ligand; CXCL: C-X-C motif chemokine ligand; GM-CSF: granulocyte-macrophage colony-stimulating factor; hsCRP: high-sensitivity C-reactive protein; ICAM: intercellular adhesion molecule; IFN: interferon; IL: interleukin; MCP: monocyte chemoattractant protein; MDC: macrophage-derived chemokine; MIP: macrophage inhibitory protein; IP-10: interferon-γ induced protein 10 kDa; SAA: serum amyloid A; TARC: thymus and activation-regulated chemokine; TNF: tumour necrosis factor; VCAM: vascular cell adhesion molecule; VEGF: vascular endothelial growth factor.

Owing to their large number, samples were processed across pairs of each plate type. Each pair of plates was processed on the same day to minimise variation between plates for each set of analytes. Although V-PLEX™ plates are specifically designed and certified to have minimal variation in assay performance between separate plates of the same lot number, additional steps were taken to minimise any potential for plate-to-plate variation to affect data analysis. First, for each plate pair, all baseline samples were included together on the same individual plate. This allowed for comparison of baseline samples between patients independent of any effect of plate-to-plate variation. Secondly, it was ensured that all samples from each individual patient were included together on the same individual plate. This allowed for comparison between study time points at the level of each individual patient independent of any effect of plate-to-plate variation in absolute quantification.

Following processing, plates were immediately analysed by electrochemiluminescence (ECL) using a MESO™ QuickPlex SQ120 (Meso Scale Diagnostics, LLC.) according to the manufacturer's instructions. Signal—concentration curves based on serial dilutions of a known supplied standard were generated for each assay to calculate the concentration of analytes in each sample using the Discovery Workbench™ software version 4.0 (Meso Scale Diagnostics, LLC.). For each assay, the upper limit of detection (ULOD) for was defined as the concentration of the highest calibrator, and the lower limit of detection (LLOD) was defined as 2.5×standard deviation above the lowest calibrator concentration. Analytes with an ECL signal corresponding to a calculated concentration above the ULOD were assigned a calculated concentration equal to the ULOD. Analytes with an ECL signal corresponding to calculated concentration below the LLOD were assigned a calculated concentration equal to the LLOD. Where a sample was duplicated on the same plate (for internal quality control purposes), the mean calculated concentration value was used for analysis.

Data Analysis

Analyses were performed according to the structure detailed below. All Cox regression models used Breslow approximation for handling of tied survival times (Themeau, 2015).
1. The quality of data was assessed, and where necessary, low quality data was excluded.
2. The distributions of data were summarised by descriptive statistics and, for continuous data, were visualised by standard methods including boxplots, Q-Q plots and histograms.
3. Where the distributions of data were skewed, appropriate transformations of data groups was performed (e.g. natural logarithmic transformation).
4. The association of each individual variable at baseline with time-to-flare was assessed by univariate Cox regression using the ‘survival’ package.
5. Baseline variables were selected based on their univariate p-value to be taken forward to a multivariate Cox regression model. For clinical and cytokine data, an elevated significance threshold (p<0.2) was used in order to reduce the risk of type II error at this preliminary stage, in keeping with established precedent (Dales and Ury, 1978: Mickey and Greenland, 1989). In comparison, a more stringent significance threshold (p<0.001) was utilised for RNAseq univariate analysis in reflection of the several log-fold greater number of variables analysed.
6. Backward stepwise variable selection based on the Akaike information criterion (AIC) using the ‘stepAIC’ function of the ‘MASS’ package was used to create a stepwise multivariate Cox regression model. The regression coefficient (equivalent to the natural logarithm of the hazard ratio) and its corresponding 95% confidence interval for each variable was visualised in a forest plot format using the package ‘forestplot’ (and its dependent packages ‘checkmate’ and ‘magrittr’).
7. Patients were dichotomised by the levels of variables measured at baseline. In the case of binary variables, patients were dichotomised simply by presence or absence of the variable at baseline. For continuous variables, thresholds were determined by receiver operating characteristic (ROC) analysis (using the ‘pROC’ package). For each continuous variable, two optimum thresholds were set to optimise negative predictive value (NPV) and positive predictive value (PPV) for flare, corresponding to biomarker thresholds for remission and flare respectively. Confidence intervals for these metrics and the area under the ROC curve (ROC_{AUC}) were calculated by bootstrapping (2000 replicates) and the DeLong procedure (DeLong et al., 1988) respectively.
8. Variables that were significantly associated with time-to-flare in the stepwise multivariate Cox regression model at the p<0.05 threshold were then combined in composite scores, weighted by their respective coefficients in multivariate Cox regression analysis.
9. Survival curves were compared between the dichotomised groups (using the ‘survminer’, and its dependent ‘ggpubr’ and ‘ggplot2’, packages) by the log-rank test as a measure of their utility in predicting time-to-flare after DMARD cessation.
10. Finally, where data allowed, the relationship between longitudinal changes in variables at the individual patient level and time-to-flare were explored.

A key assumption of Cox regression is that the hazard function for each stratum of the dataset is proportional over time—in other words, the proportional change in hazard attributable to a given variable must be constant across all time points. In a seminal paper, Schoenfeld defined residuals of the proportional hazards model that, if proportionality of hazards is true, show no significant correlation with time (Schoenfeld, 1982). Following this approach, proportionality of hazards was tested for each individual variable in univariate and stepwise multivariate Cox regression models by plotting scaled Schoenfeld residuals against time, and fitting a smoothed spline curve with four degrees of freedom using the survival package (Themeau, 2015). Proportionality of hazards was assumed if no significant association at the 0.05 threshold was observed between scaled Schoenfeld residuals versus time.

The Akaike information criterion (AIC) was used to drive stepwise backwards selection of variables in the multivariate Cox regression models. The AIC is a well-established method of nested model selection, which penalises goodness of model fit by the number of variables contained within the model (Bozdogan, 1987). The best model, displaying the optimum balance between predictive utility and number of variables, is distinguished by the lowest AIC score. In backwards stepwise selection, a set of reduced models is created by dropping each of variables in turn from the starting model. The model with the lowest AIC is then taken forward to the next round of variable selection, and the process repeated until no further reduction in AIC is possible—the model with the lowest AIC in the final step is accepted as the final stepwise model. Stepwise backwards variable selection of Cox regression models based on AIC has been previously described in analyses of differential gene expression in cancer studies, using both microarray technology (Wozniak et al., 2013) and next-generation sequencing data (Falco et al., 2016; Fan and Uu, 2016).

### Example 2—Clinical Data Results

Baseline clinical variables that are predictive of sustained DFR versus flare following DMARD cessation are identified herein.

Patient Recruitment and Outcomes

Of the 44 patients who discontinued DMARD therapy, 23 (52%) were classified as experiencing an arthritis flare, 20 (45%) maintained DFR and 1 (2%) was effectively lost to follow-up and was analysed as being censored in remission after 69 days follow-up (FIG. 2).

Survival Analysis

The association between baseline clinical variables and time to flare following DMARD cessation was analysed by univariate Cox regression for all 44 patients who stopped DMARDs (see Table below).

| | | | | Univar- |
|---|---|---|---|---|
| | | | 95% CI | iate |
| Variable | B | HR_{flare} | HR_{flare} | p-value |
| Double seropositive | 0.982 | 2.67 | 1.17-6.09 | 0.019 |
| Symptom duration prior to | 0.036 | 1.04 | 1.00-1.07 | 0.032 |
| first rheumatology review | | | | |
| (months) | | | | |
| Current number of DMARDs | 0.704 | 2.02 | 1.06-3.86 | 0.033 |
| Cumulative number of | 0.401 | 1.49 | 0.98-2.27 | 0.060 |
| DMARDs since diagnosis | | | | |
| Months since last change in | −0.015 | 0.98 | 0.97-1.00 | 0.067 |
| DMARD therapy | | | | |
| Male sex | 0.763 | 2.14 | 0.93-4.96 | 0.075 |
| ACPA positive | 0.752 | 2.12 | 0.90-5.01 | 0.087 |
| Current hydroxychloroquine | 0.752 | 2.12 | 0.89-5.04 | 0.089 |
| Baseline ACR/EULAR | −0.692 | 0.50 | 0.22-1.14 | 0.098 |
| Boolean remission | | | | |
| Baseline VASₚₐₜᵢₑₙₜ | 0.032 | 1.03 | 0.99-1.07 | 0.100 |
| RhF positive | 0.698 | 2.01 | 0.85-4.77 | 0.113 |
| Months from first | 0.007 | 1.01 | 1.00-1.02 | 0.141 |
| rheumatology review to | | | | |
| starting first DMARD | | | | |
| Current methotrexate | 1.422 | 4.14 | 0.56-30.84 | 0.165 |
| Weekly alcohol unit intake | 0.033 | 1.03 | 0.99-1.08 | 0.167 |
| Disease duration (years) | 0.034 | 1.03 | 0.99-1.09 | 0.172 |
| Baseline 28 TJC | −0.700 | 0.50 | 0.16-1.53 | 0.222 |
| Either RhF or ACPA positive | 0.612 | 1.84 | 0.68-4.99 | 0.229 |
| Months since last steroid | −0.011 | 0.99 | 0.97-1.01 | 0.252 |
| Ever smoker | −0.405 | 0.67 | 0.29-1.52 | 0.334 |
| Baseline DAS28-ESR | 0.579 | 1.79 | 0.42-7.67 | 0.436 |
| remission | | | | |
| Baseline CRP (mg/L) | 0.055 | 1.06 | 0.92-1.21 | 0.440 |
| Baseline HAQ-DI | −0.295 | 0.74 | 0.32-1.74 | 0.495 |
| Baseline ESR (mm/hr) | 0.009 | 1.01 | 0.98-1.04 | 0.526 |
| Age (years) | 0.011 | 1.01 | 0.97-1.05 | 0.569 |
| Current smoker | −0.314 | 0.73 | 0.17-3.12 | 0.671 |
| Current sulfasalazine | −0.109 | 0.90 | 0.35-2.28 | 0.819 |
| Baseline DAS28-CRP | 0.080 | 1.08 | 0.45-2.60 | 0.858 |
| Baseline 28 SJC | 0.049 | 1.05 | 0.54-2.02 | 0.885 |
| Baseline DAS28-ESR | 0.019 | 1.02 | 0.63-1.64 | 0.938 |

Association of clinical variables with occurrence of arthritis flare following DMARD cessation by univariate Cox regression. For continuous variables, hazard ratios (HR) and the Cox regression coefficients (B) are presented for a 1 unit change in that variable. P values calculated by the Wald test. HCQ: hydroxychloroquine; MTX: methotrexate; SFZ: sulfasalazine.

Proportionality of hazards was assessed for each univariate variable by correlation of scaled Schoenfeld residuals with transformed flare-free survival time (see methods above). No significant departure from proportional hazards was observed for any of the variables.

The 15 variables with a univariate value <0.2 were then entered simultaneously in a multivariate Cox regression model. Stepwise backward selection based on AIC was then performed using the same variables to fit a stepwise Cox regression model. After 6 rounds of selection, variables remained in this stepwise model (see Table below).

| Variable | B | HR_{flare} | 95% CI | p value |
|---|---|---|---|---|
| Months from first rheumatology | 0.034 | 1.03 | 1.01-1.06 | 0.008 |
| review to starting first DMARD | | | | |
| RhF positive | 1.629 | 5.10 | 1.48-17.6 | 0.010 |
| ACPA positive | 1.589 | 4.90 | 1.36-17.7 | 0.015 |
| Baseline ACR/EULAR Boolean | −1.126 | 0.32 | 0.12-0.90 | 0.031 |
| remission | | | | |
| Current methotrexate | 2.435 | 11.41 | 1.25-104 | 0.031 |
| Months since last change in | −0.025 | 0.98 | 0.95-1.00 | 0.034 |
| DMARD therapy | | | | |
| Disease duration (years) | −0.127 | 0.88 | 0.76-1.02 | 0.092 |
| Male sex | 0.975 | 2.65 | 0.80-8.73 | 0.109 |
| Symptom duration prior to first | 0.042 | 1.04 | 0.98-1.11 | 0.158 |
| rheumatology review (months) | | | | |

Association of clinical variables with occurrence of arthritis flare following DMARD cessation, using a backwards stepwise multivariate Cox regression model. For continuous variables, hazard ratios and the Cox regression coefficients (B) are presented for a 1 unit change in that variable. P values calculated by the Wald test.

Proportionality of hazards was again assessed for each variable in the final stepwise multivariate Cox regression model. A significant departure from proportional hazards was observed only for current methotrexate use (p=0.04), though this was only notable for a single outlier with no discernible trend in the remainder of the data. The global Schoenfeld test was non-significant (p=0.49), indicating proportionality of hazards for the model as a whole.

Four variables were associated with shorter time-to-flare at the 5% significance level, namely: ACPA positivity. RhF positivity, current methotrexate and time from diagnosis to commencement of first DMARD. In contrast, fulfilment of ACR/EULAR Boolean remission criteria and time since last change in DMARD therapy were associated with a longer time-to-flare.

Composite Clinical Biomarker Score

Values of the six variables that were significantly (p<0.05) associated with time-to-flare at in the multivariate stepwise Cox regression model were multiplied by their respective coefficients in the model and then summed to create composite scores. The predictive value of all 63 possible combinations of these variables to predict flare and remission following DMARD cessation was then compared by area under the receiver-operating characteristic curve (ROC_{AUC}). The ten composite scores with the highest ROC_{AUC} are listed in the Table below.

| | | ACR/EULAR | DMARD | | DMARD | |
|---|---|---|---|---|---|---|
| RhF | ACPA | Boolean | change | Current | commencement | |
| positive | positive | remission | (months) | methotrexate | (months) | ROC_{AUC} |
| x | ✓ | ✓ | ✓ | ✓ | X | 0.850 |
| ✓ | ✓ | ✓ | ✓ | ✓ | X | 0.848 |
| x | X | ✓ | ✓ | ✓ | X | 0.837 |
| ✓ | X | ✓ | ✓ | ✓ | X | 0.833 |
| ✓ | ✓ | ✓ | ✓ | ✓ | ✓ | 0.805 |
| X | ✓ | ✓ | ✓ | ✓ | ✓ | 0.798 |
| X | ✓ | X | ✓ | ✓ | x | 0.787 |
| ✓ | X | X | ✓ | ✓ | x | 0.786 |
| ✓ | X | ✓ | ✓ | ✓ | ✓ | 0.782 |
| ✓ | ✓ | ✓ | ✓ | x | ✓ | 0.777 |

The top ten clinical composite scores ranked by ROC_{AUC}. Variables included within each score are indicated in green, and those excluded are indicated in red.

Optimal performance was observed for a four-variable composite score incorporating ACPA positivity. ACR/EULAR Boolean remission, time since last change in DMARD therapy, and current use of methotrexate (Formula below).
Formula—Composite clinical biomarker score. Values for binary variables, as indicated by the square brackets, are only added to the equation if the variable is present.

Clinical score=0.654[ACPA positive]+1.130[current methotrexate]-0.521[ACR/EULAR Boolean remission]−0.012(months since last DMARD change)

The composite clinical score performed well its ability to discriminate flare versus remission following DMARD cessation, with a total area under the ROC curve (ROC_{AUC}) of 0.85 (95% Cl 0.73-0.97). Optimal thresholds were determined to maximise detection of flare (1.82) and remission (0.51). Both thresholds performed well in the study population, with a positive predictive value (PPV) of 0.90 (95% Cl 0.78-1.00) for the flare threshold, and a negative predictive value (NPV) of 0.87 (95% Cl 0.69-1.00) for the remission threshold (Table below).

| Positive test | | | | |
|---|---|---|---|---|
| threshold | Sensitivity | Specificity | PPV | NPV |
| Flare | 0.78 | 0.90 | 0.90 | 0.78 |
| (>1.82) | (0.61-0.91) | (0.75-1.00) | (0.78-1.00) | (0.67-0.91) |
| Remission | 0.91 | 0.60 | 0.72 | 0.87 |
| (>0.51) | (0.78-1.00) | (0.40-0.80) | (0.62-0.85) | (0.69-1.00) |

Predictive utility of the clinical composite clinical score in predicting flare following DMARD cessation, with a positive test defined by either flare or remission thresholds. NPV: negative predictive value; PPV: positive predictive value.

Baseline Predictors of Drug-Free Remission

Of the six variables significantly associated with time-to-flare in the multivariate stepwise Cox regression model, four had discriminatory value in a composite score for predicting flare and remission in the six months after DMARD cessation. ACPA and RhF positivity were predictive of increased likelihood of flare; and ACR/EULAR Boolean remission and months since last DMARD change were predictive of increased likelihood of remission.

ACR/EULAR Boolean Remission

A notable finding of this study is that fulfilment of ACR/EULAR Boolean remission at baseline is associated with a 3-fold lower risk of arthritis flare in the six months following DMARD cessation. Given the relatively new introduction of this remission definition, data surrounding its utility in the prediction of DFR following DMARD cessation remains scarce. The only study to have previously addressed this issue is the RETRO study, which demonstrated only a non-significant trend towards lower flare rate in those satisfying ACR/EULAR Boolean remission (OR_{flare} 0.673, 95% Cl 0.211-2.144, p=0.503) (Haschka et al., 2016).

Any of the individual components that constitute ACR/EULAR Boolean remission failed to demonstrate a significant association with time-to-flare in the multivariate analysis, suggesting that it is the combination of variables within the definition that is of importance. Although not designed specifically for clinical use, a major limitation of the use of ACR/EULAR Boolean remission in clinical practice lies in its stringent VASₚₐₜᵢₑₙₜ≤ 1/10 threshold. Indeed, 10/74 (15%) of patients recruited to this study satisfied DAS28-CRP remission at baseline yet failed to achieve ACR/EULAR remission solely on grounds of VASₚₐₜᵢₑₙₜ alone (median 22/100, range 12-34). A modified Boolean remission criteria incorporating a higher VAS patient threshold (for example ≤20/100) may have greater specificity for flare without losing sensitivity.

### SUMMARY

ACR/EULAR Boolean remission is negatively associated with flare, suggesting a robust and stable clinical remission phenotype at baseline in those patients who subsequently achieved sustained drug-free remission.

### Example 3—Cytokine Results

The measurement of circulating chemokines and cytokines provides an attractive approach to the development of biomarkers of DFR in RA. Suitable biological samples (serum or plasma) can be obtained with simple venepuncture and require only a single centrifugation step before suitable for long-term freezer storage. Furthermore, many laboratory assays for the sensitive and specific detection of a wide range of human cytokines are already in widespread research and clinical use. A robust serum-based circulating cytokine/chemokine biomarker of DFR would thus be ideally placed for translation to a high-throughput assay suitable for use in clinical practice.

Surprisingly little research has been conducted to date that explores circulating serum predictors of DFR in RA beyond acute-phase markers. Nevertheless, available tools such as the multi-biomarker disease activity (MBDA) assay have been developed for the measurement of disease activity. It is possible that such measures could overlook more nuanced mediators that are not directly involved in the acute phase response, but nonetheless play crucial roles maintaining the balance between sustained DFR and arthritis flare.

Cytokine/chemokine biomarkers are identified herein that, when measured immediately prior to DMARD cessation, differentiate those patients who subsequently developed arthritis flare versus those who remained in sustained DFR. The longitudinal change in circulating cytokine/chemokine profile in both flare and remission groups are also explored, providing insights to the underlying immunobiology of flare and DFR.

Quality Control

In this study, commercially available multiplex electrochemiluminescence assays (MesoScale Discovery) were used for the detection of 39 chemokines and cytokines (see methods above). These kits benefit from both a highly specific antibody-based capture mechanism, together with robust quality control mechanisms to ensure reliable assay performance.

Sample Collection

Of the 44 patients who stopped DMARDs, baseline samples from 43 patients were available for analysis. Serum was available for 29/42 month one visits and for 15/19 patient-requested unscheduled visits. Serum was collected for all month 3 and month 6 visits. Serum was available at the time of flare for 22/23 patients who experienced an arthritis flare.

Time from blood draw to centrifugation was recorded for 133/136 serum samples collected, with a median value of 50 (IQR: 43-87, range: 30-210) minutes.

Calibrator Coefficient of Variation

Seven manufacturer-supplied calibrator solutions containing known concentrations of analytes were prepared by serial dilution for each plate. This allowed calculation of the percentage coefficient of variation (% CV) of calculated concentration for each calibrator on each plate (Appendix G). The manufacturer states that the % CV is typically less than 20% for repeat measurements. Over the 10 individual plates there were 656 pairs of calibrator measurements, of which % CV>20 in 60 (9%) of measurement pairs. Of these measurement pairs, 37/60 (62%) were within the three lowest concentration calibrators.

Limits of Detection

A total of 6324 assay measurements (excluding calibrators and blanks) were recorded over the 10 plates. Upper and lower limits of detection (ULOD and LLOD respectively) were calculated for each assay as previously described (see methods above). No samples exceeded the ULOD, whereas 3391 (54%) had a calculated concentration below the LLOD.

Samples where the calculated concentration fell below the LLOD were assigned a calculated concentration equal to the LLOD for each respective assay.

Two IL-8 assays were included with differing ranges of detection: standard IL-8 assay and an alternative IL-8 assay (IL-8 (HA)) with a higher detection range. Concentration measurements frequently fell below the limits of detection for the IL-8 (HA) assay, and thus results from the standard IL-8 assay were used for analysis. Similarly, two IL-17A assays were included based on different isotypes of detection antibody. A greater proportion of assays fell below the LLOD for the IL-17A assay on the Th17 panel plates in comparison to the IL-17A assay on the cytokine panel plates, and thus the former was excluded from analysis.

For analyses based solely on baseline samples, 13 assays where <20% of measurements were above the LLOD were excluded. For longitudinal analysis of flare patients, six assays where all measurements were below the LLOD at both baseline and time of flare were excluded. For longitudinal analysis of remission patients, eight assays where all measurements were below the LLOD at both baseline and month six visit were excluded. The assays included within each analysis following the above quality control steps are summarised in the Table below.

| | | Inclusion of assay by analysis type | | |
|---|---|---|---|---|
| | | | Baseline to | Baseline to |
| | | Baseline | flare visit | month six visit |
| | | (all | (flare | (remission |
| MSD plate | Assay | patients) | patients) | patients) |
| Cytokine | GM-CSF | X | X | X |
| panel 1 (human) | IL-12/IL- | ✓ | ✓ | ✓ |
| | 23p40 | | | |
| | subunit | | | |
| | IL-15 | ✓ | ✓ | ✓ |
| | IL-16 | ✓ | ✓ | ✓ |
| | IL-17A | X | ✓ | ✓ |
| | IL-1α | X | ✓ | ✓ |
| | IL-5 | X | ✓ | ✓ |
| | IL-7 | ✓ | ✓ | ✓ |
| | TNF-β | X | X | ✓ |
| | VEGF | ✓ | ✓ | ✓ |
| Chemokine | Eotaxin | ✓ | ✓ | ✓ |
| panel 1 (human) | Eotaxin-3 | ✓ | ✓ | ✓ |
| | IL-8(HA) | X | X | X |
| | IP-10 | ✓ | ✓ | ✓ |
| | MCP-1 | ✓ | ✓ | ✓ |
| | MCP-4 | ✓ | ✓ | ✓ |
| | MDC | ✓ | ✓ | ✓ |
| | MIP-1α | ✓ | ✓ | ✓ |
| | MIP-1β | ✓ | ✓ | ✓ |
| | TARC | ✓ | ✓ | ✓ |
| Proinflammatory | IFN-γ | ✓ | ✓ | ✓ |
| panel 1 (human) | IL-10 | ✓ | ✓ | ✓ |
| | IL-12p70 | X | X | X |
| | subunit | | | |
| | IL-13 | X | ✓ | X |
| | IL-1β | X | ✓ | X |
| | IL-2 | X | ✓ | ✓ |
| | IL-4 | X | ✓ | X |
| | IL-6 | ✓ | ✓ | ✓ |
| | IL-8 | ✓ | ✓ | ✓ |
| | TNF-α | ✓ | ✓ | ✓ |
| Th17 panel 1 | IL-17A | X | X | X |
| (human) | IL-21 | X | X | X |
| | IL-22 | ✓ | ✓ | ✓ |
| | IL-23 | X | X | X |
| | IL-27 | ✓ | ✓ | ✓ |
| | MIP-3α | ✓ | ✓ | ✓ |
| | IL-31 | X | X | X |
| Vascular injury | CRP | ✓ | ✓ | ✓ |
| panel 2 (human) | ICAM-1 | ✓ | ✓ | ✓ |
| | SAA | ✓ | ✓ | ✓ |
| | VCAM-1 | ✓ | ✓ | ✓ |

Inclusion of assays by type of analysis.

Logarithmic Data Transformation

The distribution of calculated concentrations for each assay was typically positively skewed. To create a more even distribution of data, the natural logarithm of each analyte concentration measurement was calculated before further data analysis. A constant value (+1) was added to each analyte concentration before logarithmic transformation to allow for zero values.

Plate Equilibration

It was not possible to analyse all serum samples together on the same plate, as the total number of samples (136) exceeded the number of available wells (80) on a single plate. It was therefore necessary to process samples across pairs of each type of MSD plate. Each pair of plates was processed on the same day to minimise variation. Furthermore, all baseline samples were run together on the same plate within each plate pair, thus permitting analysis of baseline samples without any effect of plate-to-plate variation.

By replicating baseline samples where space allowed, it was attempted to keep all samples from the same participant together on the same single plate for the purposes of longitudinal analysis—however, this was not possible in all cases. Despite the manufacturer's claims of excellent reproducibility of assay performance between plates of identical lots, it was deemed necessary to apply a data equilibration procedure to minimise any potential effect upon longitudinal analyses. First, the relationship between the concentrations of each individual analyte for duplicated samples (i.e. present on both plates within a pair) was modelled by linear regression as detailed in the formula below:

linear regression equation for plate equilibration procedure: Plate₂ concentration=m*(plate₁ concentration)+c

The regression formula was then applied to each individual concentration measurement for each assay within each plate₂, thus equilibrating plate₂ with plate₁ measurements in each plate pair. Finally, to avoid any bias introduced by differing LLODs between plate pairs, the highest LLOD of either plate within each pair was applied for the purposes of longitudinal data analyses. Where a sample had been analysed on both plates, the sample measurement on the same plate as the majority of other samples for that patient was used for analysis to reduce further any bias introduced by plate-to-plate variation.

There were 11 assays where only two or fewer samples measured above the LLOD on both plates—these assays were thus excluded from plate equilibration, namely: GM-CSF, IL-1α, TNFβ, IL-12p70, IL-13, IL-1β, IL-2, IL-4, IL-21, IL-23 and IL-31. In addition, MIP1α concentration demonstrated a poor correlation between plates and was also excluded. Plate equilibration was not performed for these samples, but rather only concentrations from a single plate (i.e. excluding values from the other plate) at the level of each patient were used for analysis.

Baseline Survival Analysis

The association between baseline cytokine/chemokine levels and time-to-flare following DMARD cessation were analysed by univariate Cox regression (see Table below) for all 26 assays that passed quality control checks (see above). Proportionality of hazards was observed for all variables with the exception of ln(ICAM1+1).

| | | | | Univariate |
|---|---|---|---|---|
| Variable | B | HR_{flare} | HR_{flare} 95% CI | p value |
| ln(MCP1 + 1) | 2.212 | 9.13 | 1.97-42.32 | 0.005 |
| ln(CRP + 1) | 0.426 | 1.53 | 1.02-2.31 | 0.042 |
| ln(Eotaxin + 1) | 1.386 | 4.00 | 0.97-16.5 | 0.055 |
| ln(IL6 + 1) | 0.730 | 2.08 | 0.97-4.45 | 0.060 |
| ln(TNFa + 1) | 1.273 | 3.57 | 0.93-13.7 | 0.063 |
| ln(IP10 + 1) | 0.592 | 1.81 | 0.97-3.38 | 0.064 |
| ln(IL10 + 1) | 1.737 | 5.68 | 0.65-49.8 | 0.117 |
| ln(IL27 + 1) | 0.948 | 2.58 | 0.78-8.53 | 0.120 |
| ln(MCP4 + 1) | 0.522 | 1.69 | 0.84-3.38 | 0.140 |
| ln(IL15 + 1) | 1.572 | 4.82 | 0.56-41.6 | 0.153 |
| ln(Eotaxin3 + 1) | 0.310 | 1.36 | 0.81-2.28 | 0.238 |
| ln(VCAM1 + 1) | 0.654 | 1.92 | 0.55-6.72 | 0.306 |
| ln(IL16 + 1) | 0.694 | 2.00 | 0.52-7.65 | 0.311 |
| ln(IL7 + 1) | 0.628 | 1.87 | 0.51-6.84 | 0.342 |
| ln(IL22 + 1) | −0.502 | 0.61 | 0.20-1.87 | 0.384 |
| ln(MIP1a + 1) | −0.485 | 0.62 | 0.19-2.04 | 0.427 |
| ln(TARC + 1) | 0.283 | 1.33 | 0.64-2.76 | 0.448 |
| ln(MIP1b + 1) | 0.342 | 1.41 | 0.47-4.17 | 0.538 |
| ln(MDC + 1) | −0.376 | 0.69 | 0.15-3.07 | 0.623 |
| ln(IL8 + 1) | 0.171 | 1.19 | 0.55-2.57 | 0.664 |
| ln(IFNg + 1) | 0.074 | 1.08 | 0.73-1.59 | 0.712 |
| ln(VEGF + 1) | −0.043 | 0.96 | 0.46-1.99 | 0.909 |
| ln(MIP3a + 1) | 0.033 | 1.03 | 0.58-1.85 | 0.912 |
| ln(SAA + 1) | 0.017 | 1.02 | 0.66-1.56 | 0.940 |
| ln(ICAM1 + 1) | 0.033 | 1.03 | 0.34-3.15 | 0.954 |
| ln(IL1223p40 + 1) | 0.019 | 1.02 | 0.47-2.21 | 0.961 |

Association between the circulating concentration of cytokines/chemokines at baseline and time-to-flare following DMARD cessation, analysed by univariate Cox regression. Hazard ratios are calculated for a 1-unit change in log-transformed cytokine/chemokine concentration. B: Cox regression coefficient. P values calculated by the Wald test.

There was no statistical association between ln(ICAM1+1) and flare by univariate Cox regression (p=0.95). To confirm that this lack of association was not a spurious effect of poor survival modelling, the relationship between ln(ICAM1+1) and occurrence of flare was assessed by univariate binomial logistic regression, which also demonstrated a similar lack of association (OR_{flare} 0.66, 95% Cl 0.12-3.72, p=0.64).

Following univariate modelling, the 10 variables with a univariate p-value <0.2 were then entered to a multivariate Cox regression model, and stepwise backward variable selection based on AIC was performed. After seven rounds of selection, three variables remained in this stepwise model (Table below). Proportionality of hazards was confirmed for all three variables by the Schoenfeld residual test.

| Variable | B | HR_{flare} | 95% CI HR_{flare} | Multivariate p |
|---|---|---|---|---|
| ln(MCP1 + 1) | 2.320 | 10.2 | 2.01-51.4 | 0.005 |
| ln(IL27 + 1) | 1.464 | 4.32 | 1.17-16.0 | 0.029 |
| ln(CRP + 1) | 0.404 | 1.50 | 0.99-2.26 | 0.054 |

Association between the circulating concentration of cytokines/chemokines at baseline and time-to-flare following DMARD cessation, analysed in a backward stepwise multivariate Cox regression model. Hazard ratios are calculated for a 1-unit change in log-transformed cytokine/chemokine concentration. B: Cox regression coefficient. P values calculated by the Wald test.

The distributions of baseline ln(MCP1+1), ln(IL27+1) and ln(CRP+1) by flare status are summarised in FIG. 3. Baseline MCP1 levels were significantly higher at baseline in those patients who experienced an arthritis flare (p=0.012, unpaired Student's T-test). There was a trend towards higher baseline CRP and IL-27 in the flare group, though this was not statistically significant (p=0.121 and p=0.224 respectively).

ROC Analysis and Biomarker Thresholds

Patients were dichotomised based on their baseline levels of MCP1, IL-27 and CRP using two thresholds determined by ROC analysis optimised for the prediction of flare and remission, as discussed above. Variables were also combined to form composite scores, weighted by their respective coefficients from the stepwise multivariate Cox regression model (see Table below).

| | | | | Flare | |
|---|---|---|---|---|---|
| ln(MCP1 + | ln(IL27 + | ln(CRP + | Remission | thresh- | |
| 1) | 1) | 1) | threshold | old | ROC_{AUC} |
| ✓ | ✓ | ✓ | 29.31 | 30.54 | 0.757 |
| ✓ | ✓ | X | 23.01 | 24.695 | 0.746 |
| ✓ | X | ✓ | 18.17 | 19.29 | 0.725 |
| ✓ | X | X | 5.43 | 5.85 | 0.705 |
| X | ✓ | ✓ | 16.15 | 17.45 | 0.664 |
| X | X | ✓ | 13.68 | 14.51 | 0.654 |
| X | ✓ | X | 7.13 | 7.69 | 0.613 |

Cytokine/chemokine composite scores ranked by ROC_{AUC}. Variables included within each score are indicated in green, and those excluded are indicated in red.

Whereas MCP1 performed reasonably well in isolation, the discriminative value of IL-27 and CRP when used on their own was relatively poor. The best results were observed for composite measures, namely MCP1+CRP and MCP1+IL27, which performed best for the identification of future remission (FIG. 4) and flare (FIG. 5) respectively. A composite score encompassing all three variables did not yield any additional predictive value (see Table below).

| | ROC_{AUC} | | Sensitivity | Specificity | PPV | NPV |
|---|---|---|---|---|---|---|
| Variable | (95% CI) | Threshold | (95% CI) | (95% CI) | (95% CI) | (95% CI) |
| MCP1 + CRP | 0.73 | Flare | 0.52 | 0.84 | 0.80 | 0.59 |
| | (0.57-0.88) | (19.29) | (0.30-0.70) | (0.68-1.00) | (0.63-1.00) | (0.48-0.72) |
| | | Remission | 1.00 | 0.37 | 0.66 | 1.00 |
| | | (18.17) | (1.00-1.00) | (0.16-0.58) | (0.59-0.74) | (1.00-1.00) |
| MCP1 + IL27 | 0.75 | Flare | 0.43 | 1.00 | 1.00 | 0.59 |
| | (0.60-0.89) | (24.695) | (0.26-0.65) | (1.00-1.00) | (1.00-1.00) | (0.53-0.70) |
| | | Remission | 0.96 | 0.32 | 0.63 | 0.88 |
| | | (23.01) | (0.87-1.00) | (0.11-0.53) | (0.56-0.72) | (0.57-1.00) |
| MCP1 + IL27 + | 0.76 | Flare | 0.43 | 0.95 | 0.92 | 0.58 |
| CRP | (0.61-0.90) | (30.54) | (0.22-0.65) | (0.84-1.00) | (0.71-1.00) | (0.50-0.69) |
| | | Remission | 0.96 | 0.37 | 0.65 | 0.89 |
| | | (29.31) | (0.87-1.00) | (0.16-0.58) | (0.58-0.74) | (0.60-1.00) |

Predictive utility of cytokine/chemokine variables In predicting flare following DMARD cessation, with a positive test defined by either flare or remission thresholds. Optimum pairs of predictive metrics are highlighted in bold. NPV: negative predictive value; PPV: positive predictive value.

Sensitivity Analysis for Time to Centrifugation

Cytokines and chemokines are known to degrade in whole blood that has been left to stand for a prolonged period before centrifugation (Jackman et al., 2011). To assess whether this may have influenced the biomarker analysis, the three variables within the composite scores (i.e. ln(MCP1+1), ln(IL27+1) and ln(CRP+1)), and time from blood draw to centrifugation, were entered to a multivariate Cox regression model. In the three visits where centrifugation delay was not recorded, the median centrifugation delay across all visits was imputed. No significant association was observed between centrifugation delay (minutes) and arthritis flare (HR_{flare} 1.00, 95% Cl 0.99-1.00, p=0.465). Similar coefficients and statistical significance were observed for the three cytokine/chemokine variables as per the main analysis, demonstrating that their utility for predicting arthritis flare was not affected by inclusion of centrifugation delay within the model (see Table below). Proportionality of hazards was observed for all individual variables and the model as a whole.

| Variable | B | HR_{flare} | 95% CI | p |
|---|---|---|---|---|
| ln(MCP1 + 1) | 2.24 | 9.41 | 1.83-48.42 | 0.007 |
| ln(IL27 + 1) | 1.47 | 4.34 | 1.18-15.94 | 0.027 |
| ln(CRP + 1) | 0.37 | 1.45 | 0.97-2.18 | 0.070 |
| Centrifuge delay | 0.00 | 1.00 | 0.99-1.01 | 0.465 |
| (minutes) | | | | |

Sensitivity analysis incorporating time from blood draw to centrifugation within a multivariate Cox regression model. Hazard ratios are calculated for a 1-unit change in log-transformed cytokine/chemokine concentration, or for a 1 minute change in centrifugation delay.

Baseline to Flare Visit

Cytokine and chemokine levels were compared at baseline and flare visits in the 22 patients who both experienced a flare and had serum available at the time of flare. The statistical significance of differences between log-transformed cytokine/chemokine concentrations at baseline and flare visits was assessed by Student's paired T-test (see Table below). Four analytes demonstrated a >1.5 fold change in concentration at the 5% significance level after multiple-test correction using the Benjamini-Hochberg procedure: CRP, SAA, IL-6 and IP-10 (FIG. 6).

| | Number | Mean logₑ(FC) | | |
|---|---|---|---|---|
| | of | from baseline | Unadjusted | Adjusted |
| Variable | samples | to flare | p value | p value |
| ln(IL1223p40 + 1) | 20 | 0.206 | <0.001 | 0.008 |
| ln(CRP + 1) | 22 | 1.111 | <0.001 | 0.008 |
| ln(VEGF + 1) | 20 | 0.239 | 0.001 | 0.014 |
| ln(IL6 + 1) | 20 | 0.545 | 0.004 | 0.031 |
| ln(Eotaxin + 1) | 20 | −0.132 | 0.006 | 0.035 |
| ln(SAA + 1) | 22 | 1.071 | 0.006 | 0.035 |
| ln(IP10 + 1) | 20 | 0.447 | 0.010 | 0.043 |
| ln(IL15 + 1) | 20 | −0.095 | 0.010 | 0.043 |
| ln(MIP1α + 1) | 20 | 0.304 | 0.013 | 0.047 |
| ln(IL16 + 1) | 20 | 0.165 | 0.014 | 0.048 |
| ln(IL27 + 1) | 22 | 0.100 | 0.021 | 0.064 |
| ln(MDC + 1) | 20 | −0.066 | 0.024 | 0.067 |
| ln(Eotaxin3 + 1) | 20 | 0.248 | 0.032 | 0.082 |
| ln(ICAM1 + 1) | 22 | 0.192 | 0.081 | 0.190 |
| In(MIP3α + 1) | 22 | 0.204 | 0.108 | 0.238 |
| ln(IL1α + 1) | 20 | 0.168 | 0.124 | 0.256 |
| ln(VCAM1 + 1) | 22 | 0.077 | 0.173 | 0.336 |
| ln(IL5 + 1) | 20 | 0.034 | 0.194 | 0.356 |
| ln(TARC + 1) | 20 | −0.076 | 0.219 | 0.381 |
| ln(IL17A + 1) | 20 | 0.006 | 0.257 | 0.385 |
| ln(IL10 + 1) | 20 | 0.058 | 0.248 | 0.385 |
| ln(TNFα + 1) | 20 | 0.089 | 0.240 | 0.385 |
| ln(IL2 + 1) | 20 | −0.023 | 0.330 | 0.454 |
| ln(IL4 + 1) | 20 | 0.008 | 0.330 | 0.454 |
| ln(IL13 + 1) | 20 | −0.029 | 0.380 | 0.502 |
| ln(IL1β + 1) | 20 | 0.007 | 0.485 | 0.615 |
| ln(MCP1 + 1) | 20 | −0.024 | 0.594 | 0.726 |
| ln(IL7 + 1) | 20 | −0.022 | 0.654 | 0.771 |
| ln(MCP4 + 1) | 20 | 0.014 | 0.739 | 0.813 |
| ln(IFNγ + 1) | 20 | −0.105 | 0.732 | 0.813 |
| ln(MIP1β + 1) | 20 | −0.012 | 0.779 | 0.829 |
| ln(IL22 + 1) | 22 | 0.019 | 0.810 | 0.835 |
| ln(IL8 + 1) | 20 | 0.015 | 0.897 | 0.897 |

Change in cytokine/chemokine concentration from baseline to flare visits in those patients who experienced an arthritis flare following DMARD cessation. Statistical significance was assessed by Student's paired T-test, with multiple test-correction using the Benjamini-Hochberg procedure. FC: fold-change.

Baseline to Month Six Remission Visit

Cytokine and chemokine levels were compared at baseline and month six visits in the 19 patients who maintained DFR and had serum available at baseline and month six. The statistical significance of differences between log-transformed cytokine/chemokine concentrations at baseline and month six visits was assessed by Student's paired T-test (Table below). No analytes exceeded a >1.5 fold change in concentration at the 5% significance level (FIG. 6).

| | | Mean logₑ(fold | | |
|---|---|---|---|---|
| | Number | change) from | | |
| | of | baseline to | Unadjusted | Adjusted |
| Assay | samples | month 6 | p value | p value |
| ln(TNFα + 1) | 14 | 0.203 | 0.051 | 0.562 |
| ln(MCP1 + 1) | 14 | 0.088 | 0.064 | 0.562 |
| ln(IL15 + 1) | 14 | −0.074 | 0.077 | 0.562 |
| ln(IL1223p40 + 1) | 14 | 0.151 | 0.099 | 0.562 |
| ln(MIP3α + 1) | 19 | 0.208 | 0.100 | 0.562 |
| ln(IP10 + 1) | 14 | 0.284 | 0.121 | 0.562 |
| ln(TARC + 1) | 14 | −0.077 | 0.127 | 0.562 |
| ln(IL1α + 1) | 14 | −0.069 | 0.188 | 0.693 |
| ln(IFNγ + 1) | 14 | 0.276 | 0.216 | 0.693 |
| ln(IL5 + 1) | 14 | 0.029 | 0.259 | 0.693 |
| ln(IL10 + 1) | 14 | 0.019 | 0.260 | 0.693 |
| ln(IL7 + 1) | 14 | −0.066 | 0.294 | 0.693 |
| ln(IL27 + 1) | 19 | 0.039 | 0.309 | 0.693 |
| In(TNFβ + 1) | 14 | 0.001 | 0.336 | 0.693 |
| ln(IL2 + 1) | 14 | −0.013 | 0.336 | 0.693 |
| In(VEGF + 1) | 14 | 0.059 | 0.419 | 0.771 |
| ln(VCAM1 + 1) | 19 | 0.049 | 0.423 | 0.771 |
| ln(ICAM1 + 1) | 19 | 0.057 | 0.534 | 0.803 |
| ln(IL16 + 1) | 14 | −0.046 | 0.541 | 0.803 |
| ln(SAA + 1) | 19 | 0.116 | 0.542 | 0.803 |
| ln(IL17A + 1) | 14 | 0.013 | 0.544 | 0.803 |
| ln(MIP1β + 1) | 14 | 0.022 | 0.598 | 0.843 |
| ln(Eotaxin3 + 1) | 14 | 0.059 | 0.626 | 0.844 |
| ln(IL22 + 1) | 19 | 0.028 | 0.688 | 0.872 |
| ln(MDC + 1) | 14 | −0.017 | 0.704 | 0.872 |
| ln(IL6 + 1) | 14 | 0.003 | 0.768 | 0.912 |
| ln(MCP4 + 1) | 14 | 0.022 | 0.794 | 0.912 |
| ln(MIP1α + 1) | 14 | −0.018 | 0.862 | 0.955 |
| ln(IL8 + 1) | 14 | 0.010 | 0.929 | 0.988 |
| ln(CRP + 1) | 19 | 0.011 | 0.968 | 0.988 |
| ln(Eotaxin + 1) | 14 | −0.001 | 0.988 | 0.988 |

Change in cytokine/chemokine concentration from baseline to month six visits in those patients who remained in remission following DMARD cessation. Statistical significance was assessed by Student's paired T-test, with multiple test-correction using the Benjamini-Hochberg procedure.

Longitudinal Change in Selected Cytokines and Chemokines

The longitudinal change in circulating concentrations of selected cytokines and chemokines was explored across all study visits where serum samples were available for analysis. Descriptive analysis with the aid of longitudinal ‘spaghetti plots’ is presented herein (FIG. 7).

Both CRP and SAA concentrations rose in the approach towards arthritis flare, and remained essentially static for patients in remission, with the exception of two remission patients who had a concomitant urinary tract and lower respiratory tract infections respectively at the time of their month 3 study visit. IL6 concentrations abruptly rose at the time of flare, and remained static for patients in remission, although many samples fell below the plate-merged LLOD. Overall, IP10 concentrations rose higher in the flare versus remission group, though there was greater heterogeneity in the individual patient trends.

There was a modest overall increase in IL-27 from baseline to flare which was not observed in patients who remained in remission, although the individual patient trends were somewhat mixed. There were no clear longitudinal trends in MCP1, in contrast to its utility for predicting flare when measured at baseline.

Monocyte Chemoattractant Protein 1 (MCP-1/CCL2)

In this study, MCP-1 is predictive of outcome following DMARD cessation, with low levels at baseline demonstrating particular discrimination for those patients who maintained DFR. This observation is biologically plausible—low circulating levels of MCP-1 could conceivably reflect lower production by stromal and immune cell mediators within the synovium, thus reducing recruitment of monocytes, T cells and NK cells to the joints.

Interestingly, no significant increase in MCP-1 concentration was observed in longitudinal analysis of patients who experienced an arthritis flare. This observation suggests a possible modulatory effect of MCP-1 at baseline, as opposed to representing a surrogate measure of disease activity. However, whether the observed low levels of circulating MCP-1 reflect a qualitative difference in immune homeostasis in those patients who maintain DFR, rather than simply a quantitative difference in subclinical synovitis, remains open to speculation and would require the study of matched synovial biopsy tissue in these patients.

Interleukin-27

In this study, circulating levels of IL-27 at baseline is predictive of outcome following DMARD cessation, with high levels at baseline demonstrating particular discrimination for those patients who experienced an arthritis flare. Furthermore, there is a significant though small increase in circulating levels of IL-27 at the time of flare (mean fold change 1.07, unadjusted p=0.021). Taken together, these results imply a positive association between circulating IL-27 and arthritis flare. This would be in keeping with pro-Th1 actions of IL-27, though it is impossible to infer causality based on these data. Indeed, higher levels of IL-27 at baseline may serve to modulate the response to upstream perturbations of the immune response following DMARD cessation rather than play a causative role per se, for example by altering the signalling of other STAT-pathway cytokines such as IL-6. The robust increase in IL-6 observed at the time of flare in this study (discussed further below) lends some support to this hypothesis. Alternatively, it is even conceivable that high IL-27 may reflect a regulatory response to pro-inflammatory mechanisms, but that this regulatory response is somehow deficient in those patients who subsequently develop an arthritis flare.

Longitudinal Cytokine Data

Longitudinal observation of trends in circulating cytokine levels provides a tantalising insight in to the mechanisms underlying the emergence of RA flare. The most striking observation was that significant changes in cytokine levels were only observed in the flare group, whereas those patients who maintained DFR demonstrated very little, if any, longitudinal change in cytokine levels. This presumably reflects the substantial dysregulation of immunity that occurs at the time of arthritis flare, which is readily detected in the peripheral circulation. In contrast, the continued immune homeostasis underlying those who maintain DFR is likely to generate more subtle signals, which may furthermore be detectable only at the individual joint level.

The acute-phase proteins CRP and serum amyloid A (SAA) demonstrated the greatest increase from baseline to time of flare (mean fold change 2.16 [adjusted p=0.008] and 2.10 [adjusted p=0.035] respectively). Taken together, the observed increased concentrations of CRP and SAA at the time of flare in this study provide biochemical evidence of a robust systemic inflammatory response in patients who experienced an arthritis flare.

Further analysis of longitudinal cytokine trends in this study yields additional insights beyond only an increase in acute-phase response. IL-6 concentration was higher at the time of flare than baseline (mean fold change 1.50, corrected p=0.031) despite many results falling below, and thus being assigned the value of, the lower limit of detection for the assay. Given this technological limitation in assay performance, it is likely that the true difference in IL-6 concentration between baseline and flare is actually greater than that observed.

The observation that IL-6 levels rise in the approach to arthritis flare in this study is in keeping with the well-established role of IL-6 in the pathogenesis of the disease. Additionally, co-stimulation of Toll-like receptor 3 (TLR3) and TLR7 pathways has been observed to lead to transcriptional synergy of IL-6 and IL-12 in mouse macrophages, an effect mediated by interaction of the transcription factors Jun B. CCAAT/enhancer-binding protein beta (C/EBPβ) and IRF1 at the IL6 and IL12b promoters (Liu et al., 2015). This effect may be of relevance given the statistically significant though small increase in circulating IL-12 at the time of flare in this study (fold change 1.15, adjusted p<0.008).

Circulating concentration of the chemokine interferon-γ inducible protein 10 (IP10, also known as CXCL10) was significantly higher at the time of flare (fold change 1.36, adjusted p=0.043). Circulating concentrations of IP-10 are higher in RA patients versus healthy controls and correlate with disease activity (Kuan et al., 2010; Pandya et al., 2017).

The observation of higher circulating levels of IP-10 at the time of flare in this study would therefore be in keeping with an active flare phenotype where activated lymphocytes are recruited to the inflamed synovium. Furthermore, the postulated role of IP-10 in amplifying the recruitment of Th1 cells may be of significance given the increased levels of the pro-Th1 cytokine IL-12 at the time of flare.

Summary

Circulating concentrations of IL-27, MCP1 and CRP at baseline were predictive of flare and remission following DMARD cessation. Longitudinal analysis demonstrated increased levels of acute phase reactants (CRP and SAA) at the time of flare, together with the pro-Th17 cytokine IL-6, and the pro-Th1 mediators IL-12 and IP-10. Taken together, one can hypothesise a potent pro-inflammatory cytokine and chemokine milieu at the time of arthritis flare, characterised potentially by dysregulated Th17 and Th1 responses. The conservative approach of this study is likely to have artificially reduced the magnitude of observed increases in cytokine concentrations, such as that observed for IL-6 for example.

### Example 4—CD4⁺ T Cell RNAseq Results

Circulating CD4⁺ T cells provide a potential cellular compartment that is both important in RA pathobiology as well as easily obtainable without the need for invasive sample collection procedures. In previous studies, a protocol was developed to isolate CD4⁺ T cells from whole blood with a high cell yield and purity (Pratt, 2011). Herein, the transcriptional profiling of circulating CD4⁺ T cells unexpectedly represents a target to identify cell-specific biomarkers of drug-free remission.

Patient Samples

CD4⁺ T cell samples were available at all baseline, month three and month six study visits. In total. CD4⁺ T cell RNA was available for sequencing for 120/154 (78%) of study visits. CD4⁺ T cell RNA was available for sequencing at the time of flare for 18/23 (78%) patients who experienced an arthritis flare and was available at month six for 15/20 (75%) patients who maintained drug-free remission.

Time from blood draw to start of CD4⁺ T cell processing, and time from start of T cell processing to freezing of T cell lysate, were recorded for 117/120 of the sequenced patient samples. The median (IQR, range) time from blood draw to start of processing was 95 (80-150, 40-388) minutes. The median (IQR, range) time from start of processing to freezing of lysate was 242 (217-285, 166-333) minutes.

The number of cells in every T cell isolate was recorded to calculate the cell yield, with a median (IQR, range) value of 2.3×10⁵ cells per ml in whole blood (1.8-3.2, 0.5-7.2). The purity of every CD4⁺ T cell isolate was confirmed by flow-cytometric analysis as described (see methods above). The median (IQR, range) percentage of CD3⁺CD4⁺ cells in patient T cell isolates was 98.9 (98.3-99.3, 95.3-99.8). The percentage of contaminating monocytes and B-cells, defined as CD14⁺ or CD19⁺ respectively, were generally low. To account for small variations in CD4⁺ T cell purity, the percentage of CD3⁺CD4⁺ cells was included as a covariate in gene expression models together with sequencing batch.

Healthy Controls

Four healthy volunteers were recruited to donate blood as a control arm for comparison with the study population. The volunteers were aged 50 years (male), 40 years (male), 31 years (female) and 31 years (female) at the time of first donation. Sampling was performed at four time points (baseline, month 1, month 3 and month 6) from all participants giving 16 healthy CD4⁺ T cell samples in total. Blood was drawn where possible between 9 am-1 pm and left to stand before processing to mimic the collection and transport of the patient samples. Time from blood draw to start of CD4 T cell processing, and time from start of T cell processing to freezing of T cell lysate, were recorded for all samples. The median (IQR, range) time from blood draw to start of processing was 67 (56-90, 28-198) minutes. The median (IQR, range) time from start of processing to freezing of lysate was 254 (231-305, 170-333) minutes. There was no significant difference in mean blood draw or processing times between healthy controls and patients (p=0.141 and p=0.538 respectively, Mann-Whitney U test).

The median (IQR, range) cell yield for healthy control T cell isolates was 3.1 (2.3-3.7, 1.2-5.0)×10⁵ cells per ml whole blood. The mean cellular yield was not significantly different between healthy controls and patients (p=0.267, Mann-Whitney U test). The median (IQR, range) percentage of CD3⁺CD4⁺ cells in healthy control T cell isolates was 98.1 (97.6-98.9, 96.8-99.2). The mean percentage CD3⁺CD4⁺ cells was not significantly different between healthy controls and patients (p=0.156, Mann-Whitney U test). The percentage of contaminating monocytes and B-cells, defined as CD14⁺ or CD19⁺ respectively, was also determined. Significantly greater mean CD19⁺ contamination was seen for healthy controls versus patients (p=0.022). The explanation for this is not immediately apparent. Where data were available, the mean yield of CD19⁺ cells in parallel PBMC isolations was not significantly different between patients and healthy controls (p=0.843, Mann-Whitney U test). This suggests that the higher CD19′ contamination in healthy T cell isolates was not due to a greater number of CD19⁺ cells in healthy control versus patient blood—however, an absolute CD19⁺ cell count in whole blood, as opposed to the CD19⁺ cell yield following density centrifugation, would be required to confirm this.

RNA Yield and Integrity

The quantity and quality of RNA in each T cell lysate was measured by gel electrophoresis using a Tapestation™ 4200 machine (Agilent). The median (IQR, range) RNA yield was 870 (660-884, 277-2275) ng per million cells lysed. Prior to sequencing, the 28S/18S ribosomal RNA (rRNA) ratio and estimated RNA integrity number (RIN^{e}) were calculated for each sample. Lower RIN^{e} and 28S/18S ratios suggest RNA degradation; the RNAseq platform manufacturer (llumina) recommends a RIN^{e}>8 and a 28S/18S rRNA ratio >2.0 for high quality RNAseq analysis. The median (IQR, range) RIN^{e} was 9.4 (9.2-9.6, 8.1-10.0). The median (IQR, range) 28S/18S rRNA ratio was 2.6 (2.5-2.7, 2.1-2.9). There was no correlation between RIN^{e} and total time from venepuncture to freezing of T cell lysate (repeated measures R²=−0.023, p=0.833), suggesting that the RNA integrity was not affected by laboratory processing time during the T cell isolation procedure. There was no significant difference in mean RIN^{e} between patients and healthy controls (p=0.104, Mann-Whitney U test).

Sequencing Quality

Samples were sequenced to a mean (range) depth of 12.3 (9.2-18.4) reads per sample. Sequencing read length was tightly clustered around the expected 75 bp, and no samples were found with adaptor contamination >0.1%. Quality of the sequencing was excellent with a mean Phred score >30 across all read positions and no read trimming was necessary.

Baseline Analyses

All analyses were performed with adjustment for RNA sequencing batch and sample CD4⁺ T cell purity (see above). Change in gene expression was considered significant if there was >1.5 fold-difference in expression between groups at the 0.05 significance level according to the moderated t-test, after false-discovery rate (FDR) correction using the Benjamini-Hochberg procedure. Where changes in gene expression failed to meet statistical significance, unadjusted p-values were used as in a secondary exploratory analysis with a post hoc significance threshold of p<0.001.

Flare Versus Healthy Control

CD4⁺ T cell gene expression was compared between baseline samples of patients who flared following DMARD cessation versus healthy controls. Three genes were differentially expressed after FDR adjustment (FIG. 9A), and a further 55 were differentially expressed using an unadjusted significance threshold of p<0.001 (FIG. 9B). The entire 58-gene list is detailed below:

| | | Unadjusted | HGNC | |
|---|---|---|---|---|
| Ensembl gene ID | Log₂FC | p-value | symbol | Description |
| ENSG00000171560 | 2.42 | 1.05E−07 | FGA | fibrinogen alpha chain * |
| ENSG00000106927 | 2.13 | 1.21E−06 | AMBP | alpha-1-microglobulin/bikunin |
| | | | | precursor * |
| ENSG00000171564 | 2.00 | 3.19E−06 | FGB | fibrinogen beta chain * |
| ENSG00000163631 | 4.07 | 8.15E−06 | ALB | albumin |
| ENSG00000182489 | −2.85 | 2.50E−05 | XKRX | Kell Blood Group Complex |
| | | | | Subunit-Related, X-Linked |
| ENSG00000198538 | −1.30 | 2.89E−05 | ZNF28 | zinc finger protein 28 |
| ENSG00000223551 | 1.87 | 3.22E−05 | TMSB4XP4 | thymosin beta 4, X-linked |
| | | | | pseudogene 4 |
| ENSG00000226029 | 0.78 | 8.16E−05 | LINC01772 | long intergenic non-protein |
| | | | | coding RNA 1772 |
| ENSG00000141622 | 1.47 | 9.25E−05 | RNF165 | ring finger protein 165 |
| ENSG00000251411 | 2.32 | 1.08E−04 | | (known processed |
| | | | | pseudogene) |
| ENSG00000197841 | −0.93 | 1.11E−04 | ZNF181 | zinc finger protein 181 |
| ENSG00000164136 | 1.25 | 1.13E−04 | IL15 | interleukin 15 |
| ENSG00000172985 | −1.15 | 1.13E−04 | SH3RF3 | SH3 domain containing ring |
| | | | | finger 3 |
| ENSG00000247311 | 1.85 | 1.18E−04 | | (novel antisense) |
| ENSG00000112139 | 6.09 | 1.35E−04 | MDGA1 | MAM domain containing |
| | | | | glycosylphosphatidylinositol |
| | | | | anchor 1 |
| ENSG00000088538 | 0.85 | 1.52E−04 | DOCK3 | dedicator of cytokinesis 3 |
| ENSG00000131080 | 1.84 | 1.68E−04 | EDA2R | ectodysplasin A2 receptor |
| ENSG00000229314 | 2.72 | 1.73E−04 | ORM1 | orosomucoid 1 |
| ENSG00000125726 | 1.70 | 1.85E−04 | CD70 | CD70 molecule |
| ENSG00000152242 | 0.60 | 2.01E−04 | C18orf25 | chromosome 18 open reading |
| | | | | frame 25 |
| ENSG00000261115 | 1.94 | 2.31E−04 | TMEM178B | transmembrane protein 178B |
| ENSG00000172349 | −0.64 | 2.52E−04 | IL16 | interleukin 16 |
| ENSG00000229473 | 1.63 | 2.60E−04 | RGS17P1 | regulator of G-protein |
| | | | | signaling 17 pseudogene 1 |
| ENSG00000185010 | 1.07 | 2.65E−04 | F8 | coagulation factor VIII |
| ENSG00000267939 | 2.31 | 2.77E−04 | | (novel lincRNA) |
| ENSG00000279148 | 1.87 | 2.77E−04 | | (known TEC) |
| ENSG00000265293 | 1.64 | 2.79E−04 | ARGFXP2 | arginine-fifty homeobox |
| | | | | pseudogene 2 |
| ENSG00000261487 | 1.59 | 3.00E−04 | | (known processed transcript) |
| ENSG00000197180 | 1.29 | 3.01E−04 | | uncharacterized protein |
| | | | | BC009467 |
| ENSG00000169398 | −0.82 | 3.29E−04 | PTK2 | protein tyrosine kinase 2 |
| ENSG00000082213 | −0.70 | 3.48E−04 | C5orf22 | chromosome 5 open reading |
| | | | | frame 22 |
| ENSG00000072110 | −0.76 | 3.82E−04 | ACTN1 | actinin alpha 1 |
| ENSG00000228382 | 1.91 | 3.96E−04 | ITPKB-IT1 | Inositol-Trisphosphate 3- |
| | | | | Kinase B intronic transcript 1 |
| ENSG00000131969 | 1.92 | 3.98E−04 | ABHD12B | abhydrolase domain |
| | | | | containing 12B |
| ENSG00000149557 | 2.77 | 4.12E−04 | FEZ1 | fasciculation and elongation |
| | | | | protein zeta 1 |
| ENSG00000259657 | 1.35 | 4.33E−04 | PIGHP1 | phosphatidylinositol glycan |
| | | | | anchor biosynthesis class H |
| | | | | pseudogene 1 |
| ENSG00000165259 | 1.94 | 4.42E−04 | HDX | highly divergent homeobox |
| ENSG00000264739 | 2.16 | 5.08E−04 | | (novel antisense) |
| ENSG00000162892 | −0.61 | 5.55E−04 | IL24 | interleukin 24 |
| ENSG00000115129 | 1.15 | 5.65E−04 | TP53I3 | tumor protein p53 inducible |
| | | | | protein 3 |
| ENSG00000244588 | 2.25 | 5.89E−04 | RAD21L1 | Double-Strand-Break Repair |
| | | | | Protein Rad21 cohesin |
| | | | | complex component like 1 |
| ENSG00000272329 | 1.85 | 6.00E−04 | | (known lincRNA) |
| ENSG00000154655 | 1.66 | 6.13E−04 | L3MBTL4 | l(3)mbt-like 4 (Drosophila) |
| ENSG00000272630 | 1.09 | 6.41E−04 | | (known lincRNA) |
| ENSG00000171115 | −0.81 | 6.78E−04 | GIMAP8 | GTPase, IMAP family |
| | | | | member 8 |
| ENSG00000205786 | 1.13 | 6.86E−04 | LINC01531 | long intergenic non-protein |
| | | | | coding RNA 1531 |
| ENSG00000278356 | 1.34 | 7.01E−04 | | (known sense intronic) |
| ENSG00000159882 | −0.67 | 7.02E−04 | ZNF230 | zinc finger protein 230 |
| ENSG00000246016 | 1.95 | 7.53E−04 | LINC01513 | long intergenic non-protein |
| | | | | coding RNA 1513 |
| ENSG00000272086 | 1.27 | 7.64E−04 | | (novel lincRNA) |
| ENSG00000271447 | −1.47 | 7.73E−04 | MMP28 | matrix metallopeptidase 28 |
| ENSG00000160318 | 3.03 | 8.05E−04 | CLDND2 | claudin domain containing 2 |
| ENSG00000271680 | −1.73 | 8.45E−04 | | (novel processed |
| | | | | pseudogene) |
| ENSG00000273598 | −1.89 | 8.69E−04 | | (novel unprocessed |
| | | | | pseudogene) |
| ENSG00000162946 | −0.65 | 8.87E−04 | DISC1 | disrupted in schizophrenia 1 |
| ENSG00000228543 | 2.14 | 9.03E−04 | | (known lincRNA) |
| ENSG00000151729 | 0.64 | 9.34E−04 | SLC25A4 | solute carrier family 25 |
| | | | | member 4 |
| ENSG00000219433 | −1.71 | 9.71E−04 | BTBD10P2 | BTB domain containing 10 |
| | | | | pseudogene 2 |

Unabbreviated list of differentially expressed genes at baseline between flare patients and healthy controls, using an unadjusted significance threshold of p < 0.001. Positive log-fold change indicates higher expression in the patient group, whereas negative log-fold change indicates higher expression in the control group. HGNC: HUGO gene nomenclature committee; lincRNA: long intergenic non-coding RNA; * = significant after FDR adjustment.

In embodiments of the invention, the levels of the one or more biomarkers may comprise expression levels of at least 1, 2, 3, 4, 5, 6, 7, 8, 9, 10, 11, 12, 13, 14, 15, 16, 17, 18, 19, 20 or more genes selected from any one or more of the genes in the Table above.

In certain embodiments, the levels of the one or more biomarkers may comprise expression levels of at least 1, 2, 3, 4, 5, 6, 7, 8, 9, 10, 11, 12, 13, 14, 15, 16, 17, 18, 19 or all of ENSG00000226029, ENSG00000112139, ENSG00000247311, ENSG00000198538, ENSG00000163631, ENSG00000171560, ENSG00000171564, ENSG00000256913, ENSG00000259657, ENSG00000265293, ENSG00000204380, ENSG00000228382, ENSG00000279148, ENSG00000214081, ENSG00000165259, ENSG00000251411, ENSG00000154099, ENSG00000261487 and ENSG00000253676.

In certain embodiments, the levels of the one or more biomarkers may comprise expression levels of at least 1, 2, 3, 4, 5, 6, 7, 8, 9, 10, 11, 12, 13, 14, 15, 16, 17, 18, 19 or all of ENSG00000106927, ENSG00000226029, ENSG00000112139, ENSG00000247311, ENSG00000198538, ENSG00000163631, ENSG00000171560, ENSG00000171564, ENSG00000256913, ENSG00000259657, ENSG00000265293, ENSG00000204380, ENSG00000228382, ENSG00000279148, ENSG00000214081, ENSG00000165259, ENSG00000251411, ENSG00000154099, ENSG00000261487 and ENSG00000253676.

Univariate Cox Regression

Standard bioinformatics pipelines analyse differential gene expression dependent on the presence or absence of a binary outcome measure. Whilst this approach benefits from many years of accumulated knowledge and refined computer packages, it is inherently underpowered in comparison to survival analysis when analysing time-to-event data. Therefore, in a further analysis, the association between gene expression at baseline and time-to-flare following DMARD cessation was analysed across all sequenced genes using univariate Cox regression. Using this approach, 19 genes were identified that were significantly associated with time-to-flare by a post hoc unadjusted p-value threshold of <0.001 (FIG. 14).

None of the genes were robust to multiple test correction, although the Benjamini-Hochberg procedure appeared particularly conservative in its correction relative to the two-way analyses (Table below). No significant departure from proportional hazards was observed for any of these 19 genes.

In preferred embodiments of the invention, the levels of the one or more biomarkers may comprise expression levels of at least 1, 2, 3, 4, 5, 6, 7, 8, 9, 10, 11, 12, 13, 14, 15, 16, 17, 18 or 19 of these 19 genes.

| | | HR_{flare} | | Unadjusted | Adjusted | HGNC | |
|---|---|---|---|---|---|---|---|
| Ensembl gene ID | HR_{flare} | 95% CI | B | p-value | p-value | symbol | Description |
| ENSG00000102362 | 4.30 | 1.93-9.58 | 1.46 | 2.04E−05 | 0.555 | SYTL4 | synaptotagmin like 4 |
| ENSG00000247033 | 1.84 | 1.33-2.54 | 0.61 | 4.11E−05 | 0.559 | | (novel antisense) |
| ENSG00000276571 | 0.40 | 0.25-0.65 | −0.91 | 7.33E−05 | 0.615 | | (novel antisense) |
| ENSG00000204965 | 0.46 | 0.31-0.68 | −0.78 | 9.04E−05 | 0.615 | PCDHA5 | protocadherin alpha 5 |
| ENSG00000241146 | 0.41 | 0.27-0.63 | −0.89 | 1.44E−04 | 0.785 | RPL7P41 | ribosomal protein L7 pseudogene 41 |
| ENSG00000250030 | 2.63 | 1.58-4.36 | 0.97 | 2.15E−04 | 0.863 | | (novel processed pseudogene) |
| ENSG00000213296 | 0.37 | 0.20-0.66 | −1.01 | 2.50E−04 | 0.863 | | (known processed pseudogene) |
| ENSG00000229619 | 5.30 | 2.05-13.7 | 1.67 | 2.72E−04 | 0.863 | MBNL1-AS1 | muscleblind-like protein 1 - antisense RNA 1 |
| ENSG00000125046 | 0.37 | 0.22-0.61 | −0.99 | 2.97E−04 | 0.863 | SSUH2 | suppressor of stomatin mutant uncoordination (ssu-2) |
| | | | | | | | homolog (C. elegans) |
| ENSG00000182489 | 0.53 | 0.38-0.75 | −0.63 | 3.17E−04 | 0.863 | XKRX | Kell Blood Group Complex Subunit-Related, X-Linked |
| ENSG00000144366 | 1.87 | 1.30-2.70 | 0.63 | 4.84E−04 | 1.000 | GULP1 | engulfment adaptor PTB domain containing 1 |
| ENSG00000237473 | 3.42 | 1.58-7.39 | 1.23 | 5.28E−04 | 1.000 | | (known lincRNA) |
| ENSG00000228010 | 0.25 | 0.11-0.56 | −1.38 | 6.02E−04 | 1.000 | | (novel antisense) |
| ENSG00000250827 | 0.47 | 0.30-0.74 | −0.76 | 7.86E−04 | 1.000 | MFSD4BP1 | major facilitator superfamily domain containing 4B |
| | | | | | | | pseudogene 1 |
| ENSG00000042286 | 10.2 | 2.42-42.6 | 2.32 | 7.94E−04 | 1.000 | AIFM2 | apoptosis inducing factor, mitochondria associated 2 |
| ENSG00000231305 | 0.24 | 0.11-0.56 | −1.41 | 7.99E−04 | 1.000 | | (known antisense) |
| ENSG00000255330 | 2.66 | 1.32-5.36 | 0.98 | 8.33E−04 | 1.000 | SOGA3 | suppressor of glucose, autophagy associated (SOGA) |
| | | | | | | | family member 3 |
| ENSG00000227070 | 2.15 | 1.41-3.28 | 0.77 | 8.47E−04 | 1.000 | | (novel antisense) |
| ENSG00000162636 | 12.9 | 2.57-64.5 | 2.56 | 9.14E−04 | 1.000 | FAM102B | family with sequence similarity 102 member B |

Association between baseline gene expression and time-to-flare following DMARD cessation by univariate Cox regression, using an unadjusted significance threshold of p < 0.001. HGNC: HUGO gene nomenclature committee. LincRIMA: long intergenic non-coding RIMA. P value calculated by the likelihood ratio test.

Multivariate Cox Regression

The 19 genes that were significantly associated with time-to-flare at unadjusted significance level of <0.001 by univariate Cox regression were entered into multivariate Cox regression model. Stepwise backward selection based on Akaike Information Criterion (AIC) was then performed in order to reduce the number of variables to a practical size for the purposes of a biomarker signature, where a lower AIC score indicates a better model (see above). After five selection steps, 14 variables remained in the preliminary backwards stepwise multivariate Cox model (Table below), with a reduction in AIC from 86.97 to 79.42. The 13 variables in this model with a p value <0.2 were then taken forward to a second round of stepwise backward selection. Although this 13 variable model had an AIC slightly greater AIC (79.81) than the 14 variable model, this could be reduced further by an additional two selection steps. This generated a final stepwise multivariate Cox regression model with 11 variables and an AIC (77.69) that was lower than the 14 variable model (Table below). Proportionality of hazards was demonstrated for all variables in both the 14-variable and 11-variable stepwise models, and for both models as a whole.

| Ensembl gene ID | B | HR_{flare} | 95% CI HR_{flare} | p |
|---|---|---|---|---|
| ENSG00000204965 | −2.80 | 0.06 | 0.01-0.58 | 0.015 |
| ENSG00000241146 | −1.90 | 0.15 | 0.03-0.71 | 0.017 |
| ENSG00000229619 | 4.48 | 88.2 | 2.05-3800 | 0.020 |
| ENSG00000228010 | −7.12 | 0.00 | 0.00-0.59 | 0.034 |
| ENSG00000125046 | −2.53 | 0.08 | 0.01-0.88 | 0.039 |
| ENSG00000162636 | 13.62 | 8.19 × 10⁵ | 1.18-5.69 × 10¹¹ | 0.047 |
| ENSG00000227070 | 4.11 | 60.64 | 1.03-3560 | 0.048 |
| ENSG00000250827 | 1.78 | 5.92 | 0.95-36.8 | 0.056 |
| ENSG00000042286 | 2.75 | 15.6 | 0.86-284 | 0.063 |
| ENSG00000247033 | 2.22 | 9.20 | 0.88-96.4 | 0.064 |
| ENSG00000276571 | −1.46 | 0.23 | 0.05-1.17 | 0.076 |
| ENSG00000237473 | 1.77 | 5.87 | 0.72-47.9 | 0.098 |
| ENSG00000182489 | 0.85 | 2.33 | 0.77-7.06 | 0.133 |
| ENSG00000102362 | −2.34 | 0.10 | 0.002-4.30 | 0.227 |

Association of baseline gene expression and time-to-flare following DMARD-cessation in a preliminary 14-variable backward stepwise multivariate Cox regression model. B: Cox regression coefficient. P-value calculated by the Wald test.

In preferred embodiments of the invention, the levels of the one or more biomarkers may comprise expression levels of at least 1, 2, 3, 4, 5, 6, 7, 8, 9, 10, 11, 12, 13 or 14 of these 14 genes.

| Ensembl gene ID | B | HR_{flare} | 95% CI HR_{flare} | p |
|---|---|---|---|---|
| ENSG00000228010 | −4.15 | 0.02 | 0.00-0.14 | 2.24E−04 |
| ENSG00000162636 | 6.97 | 1060 | 22.6-50000 | 3.88E−04 |
| ENSG00000227070 | 1.78 | 5.94 | 2.08-16.9 | 8.63E−04 |
| ENSG00000204965 | −1.69 | 0.18 | 0.07-0.52 | 1.45E−03 |
| ENSG00000229619 | 2.93 | 18.7 | 2.90-121 | 2.08E−03 |
| ENSG00000247033 | 1.18 | 3.26 | 1.53-6.98 | 2.29E−03 |
| ENSG00000125046 | −1.49 | 0.23 | 0.09-0.60 | 2.62E−03 |
| ENSG00000241146 | −1.32 | 0.27 | 0.10-0.71 | 7.96E−03 |
| ENSG00000276571 | −0.83 | 0.44 | 0.18-1.07 | 7.07E−02 |
| ENSG00000250827 | 0.71 | 2.04 | 0.89-4.69 | 9.42E−02 |
| ENSG00000042286 | 1.75 | 5.78 | 0.68-49.3 | 1.09E−01 |

Association of baseline gene expression and time-to-flare following DMARD-cessation in a final 11-variable backward stepwise multivariate Cox regression model. B: Cox regression coefficient. P-value calculated by the Wald test

In preferred embodiments of the invention, the levels of the one or more biomarkers may comprise expression levels of at least 1, 2, 3, 4, 5, 6, 7, 8, 9, 10 or 11 of these 11 genes.

ROC Analysis

Based on a p value threshold of <0.001, three variables were selected from this final stepwise multivariate model, namely: ENSG00000228010, ENSG00000162636 and ENSG00000227070. Candidate genes were explored for their biomarker utility either alone, or in combination with each other—when combined, expression values for each gene were weighted by the respective coefficient in the final stepwise Cox regression model. Patients were dichotomised for each gene score using two thresholds determined by ROC analysis optimised for the prediction of flare and remission, as previously discussed above (see Table below).

| | | | Remission | Flare | |
|---|---|---|---|---|---|
| ENSG00000228010 | ENSG00000162636 | ENSG00000227070 | threshold | threshold | ROC_{AUC} |
| ✓ | ✓ | ✓ | 59.19 | 60.55 | 0.900 |
| ✓ | ✓ | X | 75.86 | 79.4 | 0.854 |
| ✓ | X | ✓ | 39.33 | 41.17 | 0.841 |
| X | ✓ | ✓ | 0.18 | 2.69 | 0.820 |
| X | ✓ | X | 19.21 | 19.97 | 0.750 |
| ✓ | X | X | 56.38 | 59.1 | 0.743 |
| X | X | ✓ | −19.11 | −15.96 | 0.685 |

Composite scores ranked by area under the receiver operating characteristic curve (ROC_{AUC}). Variables included within each score are indicated in green, and those excluded are indicated in red

The ROC curve for each variable, together with the survival curves for dichotomised groups based on flare and remission biomarker thresholds are presented in FIG. 15. Based on overall AUC, itis clear to see that the composite scores provided greater predictive utility than any of the single gene expression variables alone. The prognostic performance of the composite scores are detailed further in the Table below.

| | ROC_{AUC} | | Sensitivity | Specificy | PPV | NPV |
|---|---|---|---|---|---|---|
| Variable | (95% CI) | Threshold | (95% CI) | (95% CI) | (95% CI) | (95% CI) |
| ENSG00000228010 | 0.90 | Flare | 0.78 | 0.95 | 0.95 | 0.79 |
| ENSG00000162636 | (0.81-0.99) | (60.55) | (0.61-0.96) | (0.85-1.00) | (0.84-1.00) | (0.67-0.95) |
| ENSG00000227070 | | Remission | 0.91 | 0.75 | 0.81 | 0.89 |
| | | (59.19) | (0.78-1.00) | (0.55-0.90) | (0.70-0.92) | (0.74-1.00) |
| ENSG00000228010 | 0.85 | Flare | 0.61 | 0.95 | 0.94 | 0.68 |
| ENSG00000162636 | (0.74-0.97) | (79.4) | (0.39-0.78) | (0.85-1.00) | (0.81-1.00) | (0.58-0.80) |
| | | Remission | 0.96 | 0.45 | 0.67 | 0.91 |
| | | (75.86) | (0.87-1.00) | (0.25-0.65) | (0.59-0.77) | (0.70-1.00) |
| ENSG00000228010 | 0.84 | Flare | 0.65 | 0.85 | 0.83 | 0.68 |
| ENSG00000227070 | (0.72-0.96) | (41.17) | (0.43-0.83) | (0.65-1.00) | (0.68-1.00) | (0.56-0.82) |
| | | Remission | 0.91 | 0.65 | 0.75 | 0.88 |
| | | (39.33) | (0.78-1.00) | (0.45-0.85) | (0.65-0.88) | (0.71-1.00) |

Predictive utility of cytokine/chemokine variables in predicting flare following DMARD cessation, with a positive test defined by either flare or remission thresholds. Optimum pairs of predictive metrics are highlighted in bold. NPV: negative predictive value; PPV: positive predictive value.

Overall, the 3-gene composite score performed the best for prediction of both flare and remission and discriminated patients with significantly different flare-free survival times (Formula below and FIG. 16).

The 3-gene composite biomarker score, based on log-transformed gene expression values:

Composite score=6.97(ENSG00000162636)+1.78(ENSG00000227070)−4.15(ENSG00000228010)−1.22(ACR/EULAR Boolean remission)

In even more preferred embodiments of the invention, the levels of the one or more biomarkers may therefore comprise expression levels of at least 1, 2 or 3 of the genes selected from ENSG0000022810, ENSG00000162636 or ENSG00000227070.

Sensitivity analysis Incorporating processing time During the laboratory isolation procedure, CD4⁺ T cells are alive until the final cell lysis step. The transcriptional profiles of the cells may therefore be influenced by the isolation procedure itself. In order to ascertain the effect of this upon the composite biomarker score, a sensitivity analysis was performed whereby the three genes from the final composite score were entered in to a multivariate Cox regression model with the addition of total time from venepuncture to freezing of CD4⁺ T cell lysate. Total processing time showed no significant association with time-to-flare, whereas the three genes remained strongly associated independent of processing time (Table below). Proportionality of hazards was demonstrated for all four variables and the model a whole.

| Variable | B | HR_{flare} | 95% CI HR_{flare} | p |
|---|---|---|---|---|
| ENSG00000162636 | 2.863 | 17.51 | 3.15 | 97.31 |
| ENSG00000227070 | 0.713 | 2.04 | 1.32 | 3.14 |
| ENSG00000228010 | −1.785 | 0.17 | 0.06 | 0.44 |
| Total time | −0.004 | 1.00 | 0.99 | 1.00 |

Sensitivity analysis incorporating genes from the final composite biomarker score, together with total time from venepuncture to freezing of cell lysate, in a multivariate Cox regression model. B: Cox regression coefficient

Baseline comparison of flare versus drug-free remission patients—discussion In the primary analysis of the gene expression data of this study, CD4⁺ T cell gene expression was compared at baseline between those patients who experienced an arthritis flare versus those who remained in DFR following DMARD cessation. Although substantial number of differentially expressed genes (DEGs) were observed, none of these were robust to multiple test correction. Using post hoc unadjusted p-value thresholds of <0.001 and <0.01, 11 and 118 genes respectively were identified as differentially expressed at the pre-specified >1.5 fold-change level. Pathway analysis of the all 118 DEGs identified 12 as functioning within a network of genes involved in cell cycle, cell death and inflammatory response processes, though with no connecting edges between these identified nodes.

Flare patients—flare versus baseline visits The comparison of CD4⁺ T cell gene expression between flare and baseline visits revealed two DEGs that were robust to multiple test correction, and 81 DEGs at the unadjusted p<0.001 threshold. These DEGs included up-regulation of genes encoding microtubular and centrosomal proteins, together with topoisomerase-IIα, all of which are known to play crucial roles in the cell cycle. The most significantly up-regulated gene by p-value was cell division cycle associated 7 (CDCA7). MKI67, which encodes the cell-surface molecule Ki-67, was also up-regulated at the time of flare in these cells.

Several genes not directly involved in the machinery of the cell cycle were also observed to be up-regulated at the time of flare, such as low-density lipoprotein receptor (LDLR), thyroid stimulating hormone receptor (TSHR), basic leucine zipper ATF-like transcription factor (BATF), and CD109. The observation of increased LDLR expression by CD4⁺ T cells at the time of flare may represent a regulatory response that could be perturbed in the patients who experience an arthritis flare.

DFR Patients—Month Six Versus Baseline Visits

Nineteen DEGs were observed at an unadjusted p threshold of <0.001, with substantially lower mean expression values than the 81 DEGs observed at the same significance threshold in the flare vs. baseline analysis. Furthermore, many of DEGs observed in this remission analysis were pseudogenes of unknown function. Functional analysis suggested downregulation of genes within a network of cell signalling and injury, though with very few edges between the identified nodes. Perplexingly, 3/9 of the protein-coding DEGs were immunoglobulin genes, which was unexpected in this CD4⁺ T cell analysis. This may represent genuine low-level CD4⁺ T cell expression of immunoglobulin-related genes.

Another unexpected observation was the downregulation of IL10 at month six versus baseline in DFR patients (log₂FC −0.89, unadjusted p 5.14×10⁻⁴). This was mirrored by a trend towards higher IL10 expression at the time of flare vs. baseline in those patients who experienced an arthritis flare (log₂FC 0.68, unadjusted p=0.004). Given the well-established role of IL-10 as a predominantly immunoregulatory cytokine of crucial importance to the function of Tr cells (Pot et al., 2011), it is surprising that its expression by CD4⁺ T cells would decrease with time in those patients who maintain DFR. Nevertheless, there are two conceivable explanations for this observation. First, IL-10 is not exclusively immunoregulatory, and its role in promoting B cell proliferation is thought to be of importance in the pathogenesis of some autoimmune diseases such as SLE (Peng et al., 2013). Thus, reducing IL-10 production by CD4⁺ T cells could help maintain remission in certain settings. Alternatively, lower expression of IL-10 could represent a gradual shift away from a regulatory Tr1-like phenotype and towards an effector state. However, the absence of any of the DEGs identified in the flare vs. baseline comparison would suggest that if this were occurring, then the CD4⁺ T cells are unlikely to be following a similar differentiation pathway to that observed in flare patients. Indeed, the functional relevance of this observed reduction in CD4⁺ T cell IL-10 production with time in the DFR patient population is hard to interpret in the absence of measures of inflammation and gene expression within the synovial compartment.

Predictive Biomarker Survival Analysis

Using a similar approach as previously, univariate followed by multivariate Cox regression was performed using the RNAseq data in order to develop a predictive biomarker of DFR and flare following DMARD cessation. Cox regression was selected as the analysis model of choice, as this allowed for use of a time-to-event outcome measure, rather than the binary grouping to flare versus DFR described above. This was anticipated to yield greater statistical power —indeed, unadjusted p values were on average 10-fold smaller for the Cox regression analysis compared to the baseline flare vs. DFR comparison. However, standard bioinformatics pipelines for survival analysis using RNAseq data do not currently exist, and adjustment for false-discovery rate using the standard method of Benjamini-Hochberg appeared particularly conservative when applied to the univariate Cox regression results. The issue of apportioning statistical significance in Cox regression analysis when using high dimensionality data has been highlighted in the published literature (Witten and Tibshirani, 2010).

Univariate Cox regression identified a similar set of genes associated with time-to-flare at the unadjusted p<0.001 threshold as compared to the standard analysis pipeline of DEGs between flare vs. DFR groups, thus providing a degree of internal validation of this approach. After stepwise backward multivariate Cox regression modelling, three genes were identified whose expression significantly associated with time-to-flare at the p<0.001 threshold: ENSG00000228010, ENSG00000162636 and ENSG00000227070. One of these genes (ENSG00000162636) encodes the protein family with sequence similarity 102 member B (FAM102B). Whilst no publications exist concerning the function of FAM102B, the paralogous FAM102A (also known as Early Oestrogen-Induced Gene 1) is known to be involved in oestrogen signalling (Wang et al., 2004) as well as osteoclast differentiation (Choi et al., 2013), and is implicated in cell membrane trafficking (Zhang and Aravind, 2010). Both ENSG00000228010 and ENSG00000227070 are predicted to be novel antisense genes, though no published data relates to their putative targets or physiological function. In a composite score, these three genes predicted flare and sustained DFR following DMARD cessation, with an ROC_{AUC} of 0.90.

In certain embodiments of the invention, the levels of the one or more may comprise expression levels of at least 1, 2 or all 3 of the genes selected from ENSG00000228010, ENSG00000162636 and ENSG00000227070.

Summary

The most illuminative results from the analysis of CD4⁺ T cell gene expression have come from the longitudinal comparison of flare visit with baseline. This analysis revealed a strong signature of upregulation of genes involved in cellular proliferation, as well as the pro-Th17 transcription factor BATF. These results provide evidence of activation of CD4⁺ T cells at the time of arthritis flare and are in keeping with a phenotype of systemic inflammation in keeping with clinical measures of increased disease activity, and the observed increase in levels of acute-phase and pro-inflammatory cytokines such as IL-6 (see above). Furthermore, comparison of baseline CD4⁺ T cell gene expression in flare patients versus healthy controls demonstrated upregulation of several genes encoding proteins known to be correlated with disease activity in RA, such as IL-15 and ORM1. This may suggest greater subclinical levels of inflammation in these patients at baseline, thus predisposing to a greater risk of arthritis flare upon DMARD cessation.

Unexpectedly, baseline expression of as few as three genes performed well in discriminating flare vs. DFR when re-applied to the same test cohort. The predictive performance of these genes in combination with clinical and cytokine/chemokine parameters will be addressed in the next Example.

### Example 5—Integrative Analysis

Results are analysed from all variable domains in order to synthesise a global predictive biomarker score to predict RA flare and sustained drug-free remission following DMARD cessation. The motivations for this integrative analysis are two-fold. First, by combining variables from different domains it is aimed to create a global biomarker score that outperforms any of the single-domain composite scores. Second, the process of variable combination can be expected to lead to variable redundancy, thus allowing for the final variable set to be smaller than the sum of the individual domain variable sets.

In performing this integrative analysis, a two-step variable reduction incorporating univariate approach is followed by stepwise backward multivariate Cox regression modelling. Receiver-operating characteristic (ROC) curve analysis is then used to further refine the variable set and select an optimum model for use as predictive biomarkers of flare and sustained remission following DMARD cessation.

Variable Selection

Before starting the process of integrative analysis, a reduced variable set was defined for exploration. The number of variables was reduced not only to avoid unnecessary and laborious downstream model reduction, but also to minimise over-fitting of the expansive data set to the relatively small study population.

Baseline variables were selected based upon their statistical significance in the domain-specific backward stepwise multivariate Cox regression models described in the previous Examples. Only those variables which were associated with time-to-flare in their respective multivariate models at an unadjusted significance threshold of p<0.05 (or p<0.001 in the case of gene expression data) were selected for integrative analysis. Thresholds were not set for the Cox regression coefficients, as these were expected to change with the merging of variable domains in the integrative analyses.

In total, 11 variables were selected for integrative analysis as detailed in the Table below.

| Domain | Variable |
|---|---|
| Clinical | RhF positive |
| | ACPA positive |
| | ACR/EULAR Boolean remission |
| | Months since last change in DMARDs |
| | Current methotrexate |
| Cytokine | ln(MCP1 + 1) |
| | ln(IL27 + 1) |
| | ln(CRP + 1) |
| CD4⁺ T cell gene expression | ENSG00000228010 |
| | ENSG00000162636 |
| | ENSG00000227070 |

The eleven baseline variables selected for integrative analysis.

Cox Regression

The association between baseline variables and time-to-flare following DMARD cessation was analysed by univariate Cox regression for the 43 patients where complete data was available (Table below). No significant deviation from proportionality of hazards was observed for any of the univariate variables. Given the univariate nature of this analysis, the coefficients and statistical significance of these variables mirror those already detailed in the previous Examples—minor discrepancies reflect the exclusion of the single patient without cytokine data.

| | | | | Unadjusted |
|---|---|---|---|---|
| Variable | B | HR_{flare} | HR_{flare} 95% CI | p value |
| ENSG00000228010 | −1.49 | 0.23 | 0.10-0.50 | 0.0003 |
| ENSG00000227070 | 0.75 | 2.13 | 1.40-3.24 | 0.0004 |
| ENSG00000162636 | 2.47 | 11.8 | 2.33-60.2 | 0.0029 |
| ln(MCP1 + 1) | 2.21 | 9.13 | 1.97-42.3 | 0.0047 |
| ln(CRP + 1) | 0.43 | 1.53 | 1.02-2.31 | 0.0421 |
| Months since last | −0.02 | 0.98 | 0.97-1.00 | 0.0471 |
| DMARD change | | | | |
| ACPA positive | 0.82 | 2.27 | 0.96-5.37 | 0.0622 |
| RhF positive | 0.77 | 2.15 | 0.91-5.11 | 0.0824 |
| ln(IL27 + 1) | 0.95 | 2.58 | 0.78-8.53 | 0.1203 |
| ACR/EULAR remission | −0.65 | 0.52 | 0.23-1.19 | 0.1223 |
| Current methotrexate | 1.46 | 4.31 | 0.58-32.1 | 0.1535 |

Association of baseline variables with time-to-flare following DMARD-cessation, as analysed by univariate Cox regression. B: Cox regression coefficient.

All 11 baseline variables were then entered in to a multivariate Cox regression model. Stepwise backward selection based on Akaike information criterion (AIC) was then performed to fit a stepwise Cox regression model (see above). After four selection steps, 7 variables remained in this stepwise model (Table below).

| | | | | Unadjusted |
|---|---|---|---|---|
| Variable | B | HR_{flare} | HR_{flare} 95% CI | p value |
| ENSG00000227070 | 1.14 | 3.12 | 1.81-5.36 | 0.00004 |
| ENSG00000228010 | −1.98 | 0.14 | 0.05-0.36 | 0.00005 |
| ENSG00000162636 | 2.82 | 16.72 | 2.24-125 | 0.00608 |
| ln(IL27 + 1) | 1.92 | 6.85 | 1.61-29.1 | 0.00915 |
| ACR/EULAR Boolean | −1.22 | 0.29 | 0.11-0.76 | 0.01205 |
| remission | | | | |
| RhF positive | 0.94 | 2.56 | 0.91-7.19 | 0.07470 |
| Current methotrexate | 1.66 | 5.28 | 0.55-50.2 | 0.14779 |

Association of baseline variables with time-to-flare following DMARD-cessation in a backward stepwise multivariate Cox regression model.

Proportionality of hazards was again assessed for each variable in the final stepwise multivariate Cox regression model. A significant departure from proportional hazards was observed only for current methotrexate use (p=0.006), though as noted before in Example 3, this was only notable for a single outlier with no discernible trend in the remainder of the data. The global Schoenfeld test was non-significant (p=0.087), indicating proportionality of hazards for the model as a whole.

Receiver-Operating Characteristic (ROC) Analysis

Five variables were significantly associated with time-to-flare a tan unadjusted significance threshold of <0.05 in the multivariate stepwise Cox regression model: ENSG00000227070, ENSG00000228010, ENSG00000162636 ln(IL27+1) and baseline ACR/EULAR Boolean remission. Values of these five variables were multiplied by their respective stepwise multivariate Cox regression coefficient and then summed to create composite scores. The predictive performance of all 31 potential combinations of these variables to predict flare and remission following DMARD cessation was then compared by area under the receiver-operating characteristic curve (ROC_{AUC}). The ten composite scores with the highest ROC_{AUC} are shown in the Table below.

| ACR/EULAR | | | | | |
|---|---|---|---|---|---|
| Boolean remission | ENSG00000162636 | ENSG00000227070 | ENSG00000228010 | ln(IL27 + 1) | ROC_{AUC} |
| ✓ | ✓ | ✓ | ✓ | ✓ | 0.963 |
| ✓ | ✓ | ✓ | ✓ | X | 0.954 |
| ✓ | X | ✓ | ✓ | X | 0.927 |
| ✓ | X | ✓ | ✓ | ✓ | 0.920 |
| ✓ | ✓ | X | ✓ | ✓ | 0.918 |
| X | ✓ | ✓ | ✓ | ✓ | 0.908 |
| X | ✓ | ✓ | ✓ | X | 0.908 |
| ✓ | ✓ | X | ✓ | X | 0.902 |
| X | X | ✓ | ✓ | X | 0.874 |
| ✓ | ✓ | ✓ | X | ✓ | 0.867 |

The top ten integrative composite scores ranked by ROC_{AUC}. Variables included within each score are indicated in green, and those excluded are indicated in red.

The composite score with the highest ROC_{AUC} included all variables; notably, the removal of In(IL27+1) resulted in only a small drop in ROC_{AUC} (Formulae below and FIG. 17).

Composite score=1.14(ENSG00000227070)+2.82(ENSG00000162636)+1.92(ln[IL27+1])−1.98(ENSG00000228010)−1.22(ACR/EULAR Boolean remission) Five-variable composite score

Composite score=1.14(ENSG00000227070)+2.82(ENSG00000162636)−1.98(ENSG00000228010)−1.22(ACR/EULAR Boolean remission) Four-variable composite score

Composite Score Predictive Performance

The sensitivity, specificity, positive predictive value (PPV) and negative predictive value of each of the three composite scores detailed above are presented in the Table below and FIG. 18, using the same threshold values as in the main analysis. A single optimum threshold for each composite score was selected manually based on minimising the ROC coordinate distance from the top left corner of the ROC curve plot.

| Composite | Threshold | ROC_{AUC} | Sensitivity | Specificity | PPV | NPV |
|---|---|---|---|---|---|---|
| score | value | (95% CI) | (95% CI) | (95% CI) | (95% CI) | (95% CI) |
| 5-variable | 37.41 | 0.96 | 0.91 | 0.95 | 0.96 | 0.90 |
| | | (0.92-1.00) | (0.78-1.00) | (0.84-1.00) | (0.87-1.00) | (0.78-1.00) |
| 4-variable | 23.16 | 0.95 | 0.91 | 0.89 | 0.92 | 0.89 |
| | | (0.88-1.00) | (0.78-1.00) | (0.74-1.00) | (0.81-1.00) | (0.77-1.00) |

Utility of the two composite scores in predicting arthritis flare following DMARD cessation. PPV: positive predictive value; NPV: negative predictive value.

Discussion

In this integrative analysis, variables from the composite biomarker scores of clinical, cytokine and RNAseq domains were combined together in a multivariate Cox regression model, with backwards stepwise selection used to create a final model with five variables: three gene expression (ENSG00000228010, ENSG00000162636 and ENSG00000227070), one cytokine (IL-27), and one clinical (ACR/EULAR Boolean remission). Whereas the role of the three gene variables remains unknown, the relevance of IL-27 and ACR/EULAR Boolean remission in the context of DFR has been identified previously.

When re-applied to the study population, this 5-variable composite score demonstrated a high predictive utility for outcome following DMARD cessation: ROC_{AUC} 0.96 (95% Cl 0.92-1.00), sensitivity 0.91 (0.78-1.00), specificity 0.95 (0.84-1.00), PPV 0.96 (0.87-1.00). NPV 0.90 (0.78-1.00). Thus, in this study population, patients with a negative test score had a 90% chance of remaining in DFR at the end of the six-month follow-up period, versus only 4% for those with a positive score. Such a score would undoubtedly be of great utility in helping guide DMARD withdrawal in the clinic and would represent a quantum leap beyond the approximately even chance of flare that would otherwise be predicted in the absence of a predictive biomarker based on outcome of the entire non-stratified study population.

Throughout the description and claims of this specification, the words “comprise” and “contain” and variations of them mean “including but not limited to” and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any foregoing embodiments. The invention extends to any novel one, or novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A method of determining the likelihood of a patient maintaining remission of rheumatoid arthritis (RA) following cessation of treatment with one or more disease-modifying anti-rheumatic drugs (DMARDs), the method comprising:
(i) detecting levels of one or more biomarkers in a sample of CD4⁺ T cells obtained from the patient; and
(ii) comparing the levels obtained in (i) with one or more reference levels;
wherein a difference in levels of the one or more biomarkers compared to the one or more reference levels is indicative of an increased likelihood of maintaining remission following the cessation of treatment with the one or more DMARDs, and wherein no difference in levels of the one or more biomarkers compared to the one or more reference levels is indicative of a decreased likelihood of maintaining remission following the cessation of treatment with the one or more DMARDs.

2. The method according to claim 1, wherein the sample of CD4+ T cells comprises purified or labelled CD4⁺ T cells and/or wherein the levels of the one or more biomarkers in the sample obtained from the patient are increased or decreased at least 1.5-fold or more as compared to the one or more reference levels.

3. (canceled)

4. The method according to claim 1, wherein:
(a) the one or more DMARDs are synthetic DMARDs optionally selected from methotrexate, leflunomide, sulfasalazine and/or hydroxychloroquine;
(b) the one or more DMARDs are biologic DMARDs optionally selected from etanercept, infliximab, adalimumab, certolizumab, golimumab, rituximab, abatacept, tocilizumab and/or sarilumab; and/or
(c) the one or more DMARDs are targeted synthetic DMARDs optionally selected from tofacitinib, baricitinib, filgotinib and/or upadicitinib.

5. The method according to claim 1, wherein:
(i) the one or more reference levels are obtained from healthy controls;
(ii) the one or more reference levels are obtained from patients maintaining remission of RA following cessation of treatment with the one or more DMARDs; and/or
(iii) the one or more reference levels are obtained from patients showing a flare or relapse of RA symptoms following cessation of treatment with the one or more DMARDs.

6. The method according to claim 1, wherein the levels of one or more biomarkers comprise:
(i) expression levels of at least one, two, three or more genes selected from Ensembl gene ID ENSG00000102362 (SEQ ID NO: 1), ENSG00000247033 (SEQ ID NO: 2), ENSG00000276571 (SEQ ID NO: 3), ENSG00000204965 (SEQ ID NO: 4), ENSG00000241146 (SEQ ID NO: 5), ENSG00000250030 (SEQ ID NO: 6), ENSG00000213296 (SEQ ID NO: 7), ENSG00000229619 (SEQ ID NO: 8), ENSG00000125046 (SEQ ID NO: 9), ENSG00000182489 (SEQ ID NO: 10), ENSG00000144366 (SEQ ID NO: 11), ENSG00000237473 (SEQ ID NO: 12), ENSG00000228010 (SEQ ID NO: 13), ENSG00000250827 (SEQ ID NO: 14), ENSG00000042286 (SEQ ID NO: 15), ENSG00000231305 (SEQ ID NO: 16), ENSG00000255330 (SEQ ID NO: 17), ENSG00000227070 (SEQ ID NO: 18) and ENSG00000162636 (SEQ ID NO: 19);
(ii) expression levels of at least one, two, three or more gene variants comprising a sequence having at least 95% homology to any one of SEQ ID Nos 1 to 19 based on nucleic acid identity over the entire length of the sequence;
(iii) expression levels of at least one, two, or three or more genes selected from SEQ ID Nos 13, 19, 18, 4, 8, 2, 9, 5, 3, 14 and 15; and/or
(iv) expression levels of at least one, two, three or more gene variants comprising a sequence having at least 95% homology to any one of SEO ID Nos 13, 19, 18, 4, 8, 2, 9, 5, 3, 14 or 15 based on nucleic acid identity over the entire length of the sequence.

7. (canceled)

8. The method according to claim 6 wherein the levels of one or more biomarkers comprise:
(i) expression levels of SEQ ID NO: 18, SEQ ID NO: 19 and SEQ ID NO: 13; and/or
(ii) expression levels of one or more gene variants comprising a sequence having at least 95% homology to SEQ ID Nos 18, 19 or 13 based on nucleic acid identity over the entire length of the sequence.

9. The method according to claim 1, wherein the method further comprises:
(i) detecting levels of one or more cytokines in a sample obtained from the patient; and
(ii) comparing the levels obtained in (i) with one or more reference levels;
optionally wherein the one or more cytokines comprise interleukin-27 (IL-27), CRP, SAA, IP-10, IL-6 and/or monocyte chemoattractant protein-1 (MCP-1) and/or the sample obtained from the patient that is used to detect the levels of one or more cytokines is serum or plasma.

10. (canceled)

11. (canceled)

12. The method according to claim 1, wherein the method further comprises determining the patient's ACR/EULAR Boolean Remission criteria (definition) as Tender joint count ≤1, patient global assessment of ≤1 on a 0-10 clinical scale, Swollen joint count ≤1 and C reactive protein ≤1 mg/dL (10 mg/L).

13. The method according to claim 1, wherein levels of the one or more biomarkers are detected by polymerase chain reaction (PCR), array, sequencing and/or immunoassay.

14. A method of treating or preventing RA in a patient undergoing treatment with one or more DMARDs, wherein:
(i) the patient has been identified as having an increased likelihood of maintaining remission of RA following cessation of treatment with one or more DMARDs according to claim 1, and treatment with the one or more DMARDs is reduced or ceased; or
(ii) the patient has been identified as having a decreased likelihood of maintaining remission of RA following cessation of treatment with one or more DMARDs according to claim 1, and treatment with the one or more DMARDs is maintained or increased.

15. A method of treating or preventing RA in a patient undergoing treatment with one or more DMARDs, wherein the method comprises:
(i) determining the likelihood of the patient maintaining remission of RA following cessation of treatment with one or more DMARDs according to claim 1; and
(ii)
(a) if the individual has an increased likelihood of maintaining remission of RA following cessation of treatment with one or more DMARDs according to claim 1, reducing or ceasing treatment with the one or more DMARDs; or
(b) if the individual has a decreased likelihood of maintaining remission of RA following cessation of treatment with one or DMARDs according to claim 1, maintaining or increasing treatment with the one or more DMARDs.

16. A therapeutic agent for use in a method of treating or preventing RA in a patient, wherein the patient has been identified as having a decreased likelihood of maintaining remission of RA following cessation of treatment with the one or more DMARDs according to claim 1, and the therapeutic agent is the one or more DMARDs.

17. An assay comprising:
(i) purifying or labelling CD4⁺ T cells from a sample obtained from a patient having or suspected of having RA;
(ii) detecting levels of one or more biomarkers in the purified or labelled CD4⁺ T cells; and
(iii) comparing the levels obtained in (ii) with one or more reference levels;
wherein the levels of one or more biomarkers comprise:
(a) expression levels of at least one, two, three or more genes selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 and SEQ ID NO: 19; and/or
(b) expression levels of at least one, two, three or more gene variants comprising a sequence having at least 95% homology to any one of SEQ ID Nos 1 to 19 based on nucleic acid identity over the entire length of the sequence.

18. The assay according to claim 17, wherein the levels of one or more biomarkers comprise:
(i) expression levels of at least one, two, or three or more genes selected from SEQ ID Nos 13, 19, 18, 4, 8, 2, 9, 5, 3, 14 and 15;
(ii) expression levels of at least one, two, three or more gene variants comprising a sequence having at least 95% homology to any one of SEQ ID Nos 13, 19, 18, 4, 8, 2, 9, 5, 3, 14 or 15 based on nucleic acid identity over the entire length of the sequence;
(iii) expression level of SEO ID NO: 18, SEO ID NO: 19 and SEQ ID NO: 13; and/or
(iv) expression levels of one or more gene variants comprising a sequence having at least 95% homology to SEO ID Nos 18, 19 or 13 based on nucleic acid identity over the entire length of the sequence.

19. (canceled)

20. The assay according to claim 17, wherein:
(i) the levels of the one or more biomarkers are increased compared to the one or more reference levels;
(ii) the levels of the one or more biomarkers are increased by at least 1.5 fold or more compared to the one or more reference levels;
(iii) the one or more reference levels are obtained from patients maintaining remission of RA following cessation of treatment with the one or more DMARDs; and/or
(iv) the one or more reference levels are obtained from patients showing a flare of RA symptoms following the cessation of treatment with one or more DMARDs.

21. (canceled)

22. (canceled)

23. The assay according to claim 17, wherein
the assay further comprises:
(i) detecting levels of one or more cytokines in a sample obtained from the patient; and
(ii) comparing the levels obtained in (i) with one or more reference levels;
optionally wherein the one or more cytokines comprise IL-27, CRP, SAA, IP-10, IL-6 and/or MCP-1 and/or the sample obtained from the patient that is used to detect the levels of one or more cytokines is serum or plasma.

24. (canceled)

25. (canceled)

26. The assay according to claim 17, wherein the method further comprises determining the patients ACR/EULAR Boolean Remission criteria (definition) as Tender joint count ≤1, patient global assessment of ≤1 on a 0-10 clinical scale, Swollen joint count ≤1 and C reactive protein ≤1 mg/dL (10 mg/L).

27. The assay according to claim 17, wherein levels of the one or more biomarkers are detected by polymerase chain reaction (PCR), array, sequencing and/or immunoassay.

28. A kit comprising reagents to carry out the method according to claim 1 or the assay according to claim 17, wherein the kit comprises one or more agents capable of specifically binding to the one or more biomarkers within CD4⁺ T cells in a sample obtained from the patient.

29. The kit according to claim 28, wherein
(i) the one or more agents are labelled;
(ii) the one or more agents are primers; and/or
(iii) the kit comprises agents capable of specifically binding to SEO ID NO: 19 and SSEQ ID NO: 13.

30.-34. (canceled)
